(19) United States
(12) Patent Application Publication
(10) Pub. No.: US 2008/0028478 A1
(43) Pub. Date: Jan. 31, 2008

(54) Method for Genetic Improvement of Terminal Boars

(76) Inventors: Samuel T. Buttram, Ellisville, MO (US); Michael M. Lohuis, Des Peres, MO (US); Youping Gu, Chestefield, MO (US); Christina M. Wagner, Columbia, IL (US)

Correspondence Address: HOWREY LLP, C/O IP DOCKETING DEPARTMENT, 2941 FAIRVIEW PARK DRIVE SUITE 200, FALLS CHURCH, VA, 22042, US

(21) Appl. No.: 10/565,548

(22) PCT Filed: Jul. 26, 2004
(86) PCT No.: PCT/US04/24168

§ 371 Date: Jan. 23, 2006

Related U.S. Application Data
(60) Provisional application No. 60/492,395, filed on Aug. 4, 2003.

(51) Int. Cl. A01K 67/027

(52) U.S. Cl. 800/17; 800/21

(57) ABSTRACT

The methods provided herein relate to swine production. Specifically the instant invention provides methods for the production of terminal parent animals (i.e. terminal sires and terminal dams) for use in swine production herds. Also provided are methods for quickly and efficiently introducing and/or fixing one or more desirable traits or alleles in a swine herd. Alternatively, the present methods may be employed to eliminate a particular undesirable trait or gene. The invention also provides for herds that have been developed using any of the methods described herein. The invention also provides for the use of embryo transfer, including markerassisted embryo transfer to facilitate the transfer of genetic material, particularly when it is desirable to maintain the specific-pathogen free status of a herd.

This application claims the benefit of U.S. provisional application No. 60/492,395, filed Aug. 4, 2003, which is hereby incorporated by reference.

### BACKGROUND OF THE INVENTION

1. Field of the Invention

The present invention relates generally to the field of improving porcine (pig) genetics, at both the individual animals and herds levels. Among the various embodiments, it particularly concerns a method for improving and producing terminal sires so that these boars have improved genetic merit as compared with the average herd animal.

2. Description of Related Art

Prior to the widespread use of artificial insemination (AI) in the swine industry, it was common practice to produce crossbred boars to be used as terminal sires for producing market hogs. This was done because the heterosis exhibited by crossbred boars was important for improving their breeding performance and fertility as terminal sires, when used for natural breeding service. Also, a much larger number of terminal sires were needed prior to the introduction AI than are needed today (the female to male ratio of was about 18:1 with natural service versus 200:1 with AI) and the cost of producing crossbred boars was lower than that of purebred boars. As a result the preferred method of terminal boar production was to maintain two relatively small purebred “genetic nucleus” (GN) herds (˜200 to 300 sows each) that were used to produce male and female replacements for a relatively larger target herd (˜2000 to 5000 sows). The males and females in the target herd were crossed to produce the crossbred terminal sires.

The disadvantages of this breeding program included: 1) the terminal boars were always genetically inferior to the GN herds because it was necessary to use the genetically superior males and females as replacements for the GN herd to maximize rate of genetic progress, and use the animals of lower genetic rank as replacements for the target herd; 2) genetic variability among terminal market hogs was considerable because crossbred terminal boars were often mated to females of a different genetic line; and 3) the progeny performance of the terminal sires was determined by the average genetic merit of the two purebred lines, which usually resulted in terminal boars that were inferior to one or other of the two purebred lines, unless the two purebred lines had the same genetic merit.

As AI became widely accepted, the need for heterosis in the terminal boar declined because semen is routinely checked for fertility before it is used. Moreover, it is no longer necessary to have boars that are aggressive natural breeders. Also because of the larger impact of a single AI sire versus a natural service sire (˜10 times as many progeny produced per sire), genetic merit has become relatively more important than cost per boar.

Therefore, most pig breeding-stock suppliers have moved to a breeding system that uses the single best purebred line available for a particular terminal boar product. This usually involves the production of terminal boars from a single GN herd that is sized according to the projected needs for sale (˜500 sows for example). This reduces genetic lag and genetic variability of the terminal market hog (the first two disadvantages described for crossbred terminal boars) and completely eliminates the need for two purebred lines (the third disadvantage described for crossbred terminal boars). However, given that the top boars produced in the GN are retained as GN replacements and only the remaining, slightly inferior, boars are sold as terminal sires, there is always some genetic lag using this approach. Moreover, the need to manage inbreeding through the use of multiple sires (˜20-24/year) in the GN herd makes it difficult to make rapid increases in the occurrence of alleles that might exist at very low frequencies in the population. These increases are problematic because rapid shifts in gene frequencies in the terminal sire population are desirable in the short run; however such changes put the GN herd at great risk in the longer term.

Owing to the rapidly growing and improving field of genomics, there is a need for a means of using newly available genotypic information to improve the development of commercial swine products. Such a means must allow for the rapid genetic improvement in desirable traits in a swine population (e.g., target herd) without jeopardizing the potential for long-term genetic improvement. Such a method would need to provide a means for quickly and efficiently maximizing the usefulness of new understanding regarding the function of various genes and/or combination of genes; while at the same time optimizing the use of both phenotypic and genotypic information. The instantly disclosed invention solves previously existing problems by providing a method for the rapid genetic change of a target herd, based on phenotypic and genotypic information about both the target herd and the GN herd, while maintaining a more balanced approach to selection in the GN herd.

### SUMMARY OF THE INVENTION

The instantly disclosed invention solves the deficiencies associated with previously available methodology (i.e., the problems of the genetic lag of commercial terminal boars relative to the GN herd, genetic variability of the terminal market hog, and the to inability to make rapid changes in gene frequencies of commercial terminal boars) by allocating a small purebred GN herd (˜200 sows) for the purpose of maximizing rate of genetic progress over the long term and, simultaneously, creating a second purebred sow herd (target herd) of the same genetic line that will be mated to a small number of elite sires (e.g., ˜1 to 3 sires/generation) produced from the GN.

Because these elite sires are genetically equivalent to the top sires used in the GN, the average genetic merit of the target herd will be superior to that of the GN herd, thereby eliminating genetic lag. The smaller the number of elite sires used to breed the target herd, the greater the potential genetic impact. The genetic superiority of the target herd can be further enhanced by the selection of female progeny from the elite sires as replacements to be used in the target herd. Genetic diversity and inbreeding can be controlled in the GN herd through the use of multiple sires as described above, making it relatively easy to select a sire or sires to be used in the target herd each generation that is/are not closely related to the one(s) used in the previous generation.

Thus, the constraint imposed by inbreeding concerns is essentially eliminated in the target herd as long as only females are retained as replacements in the target herd and unrelated boars are brought in from the GN herd. This frees the breeder to use as few as one boar to mate an entire generation of females, if desired, to make rapid changes of gene frequencies in the population (to, for example, meet short term demands of the marketplace) while simultaneously maintaining long-term breeding objectives in the GN herd. Another advantage of using a small number of sires is that genetic uniformity will be further improved relative to the more traditional approach.

The instant invention is directed to methods for producing improved swine genetics, both at the level of the individual animal and at the herd level, including the GN herd. One particular embodiment of the instant invention provides for the production and improvement of genetics in terminal swine parents.

Other embodiments of the instant invention provide methods for producing terminal swine parents and/or replacement animals, wherein the methods comprises the steps of: a) selecting a trait or traits for which improvement is desired; b) using semen from an elite sire which has the germplasm required to improve the traits in the progeny to breed females in a target herd; c) producing offspring from the females bred with the elite sire semen and/or embryos and selecting the offspring exhibiting improved germplasm for use as terminal sires or replacement animals.

In various aspects of the invention the trait(s) sought to be improved are selected for the presence of desirable characteristics, including but not limited to: the presence or absence of specific genes and/or alleles, health traits, reproduction traits, meat quality traits and efficient growth traits. Animals may be selected by any suitable means; for example using computer programs or other means for recording parentage/pedigree and selecting the most suitable pairings.

Using the methods of the instant invention selected genetic improvement(s) can be made at any or all levels of swine production. That is, improvements may be made in the production swine (SP) herd, the genetic nucleus (GN) herd and/or a target herd, either independently or concurrently. These herds can be located and operated on farms internal or external to breeding company facilities (e.g. GN, target herd, and/or SP herds can be owned and operated at customer locations with genetic expertise supplied by the breeding company).

The present invention also provides for GN herds, target herds, and/or SP herds that have been produced or genetically modified through the use of the methods described herein.

### BRIEF DESCRIPTION OF THE DRAWINGS

The described drawings form part of the present specification and are included to further demonstrate certain aspects of the present invention. The invention may be better understood by reference to one or more of these drawings in combination with the detailed description of specific embodiments presented herein.

FIG. 1: This figure provides a schematic overview of a traditional swine breeding strategy. The drawing shows the traditional use of a GN herd as well as the definition of High and Average Health locations.

FIG. 2: The figure provides a schematic overview of one embodiment of the instant invention. The drawing shows the use of an “Elite Sire” to create terminal (EBX) boars and the relationship among the GN and target herds (in this case the target herd is the PN herd).

FIG. 3: This drawing contrasts traditional GN herd terminal boar production with the instant invention (the Super Sire Concept). The drawing also shows the relationship among the GN herd and the “production nucleus” or “target” herds as wells as use of the elite sires.

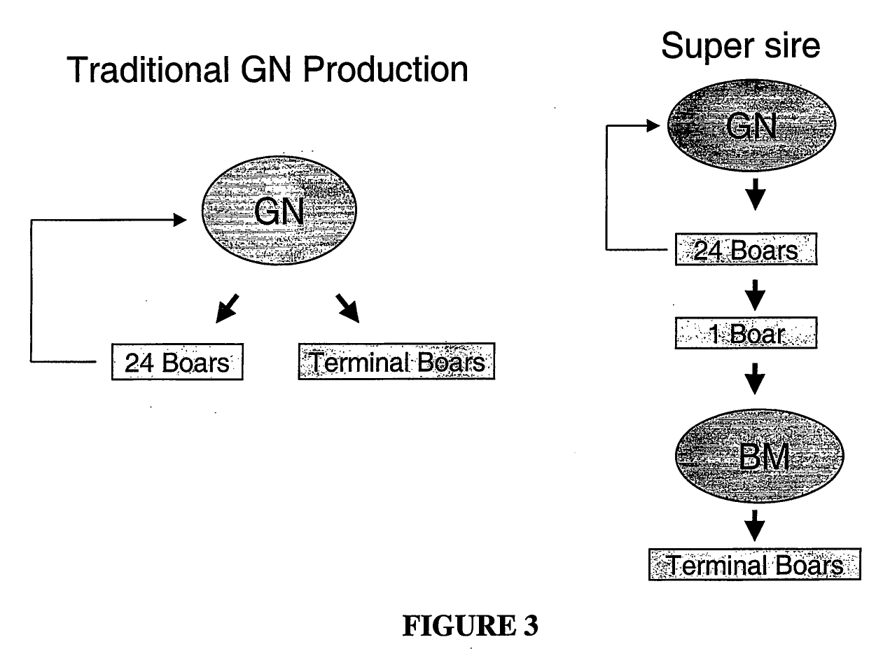

FIG. 4: Drawing depicting the multi-generational breeding program used in the swine production industry. “GN” stands for genetic nucleus herd, “GGP” stands for great grand parent herd, “GP” stands for grand parent herd, “SP” stands for swine production herd, and “commercial” refers to the herds used to produce the swine used for commercial consumption.

FIG. 5: Shows the proportion of boars with rare and very rare alleles resulting from selection in the target (PN) and GN herds.

FIG. 6: Shows the proportion of boars with rare and very rare alleles resulting from selection in the target (PN) herd only.

FIG. 7: A schematic representation of the reproductive tract of a female pig. The representation indicates the regions where semen is deposited during standard artificial insemination, intrauterine insemination, and deep intrauterine insemination.

FIG. 8: A photograph of typical cervical catheters used in standard artificial insemination.

FIG. 9: A photograph of examples of the type of catheters used in intrauterine artificial insemination.

FIG. 10: A photograph of an example of a catheters suitable for use for deep intrauterine artificial insemination.

FIG. 11: Schematic representation of method for genetic improvement of terminal sires known as THE CHOICE ADVANTAGE SYSTEM^{SM}.

FIG. 12: Schematic representation of the use embryo washing and embryo transfer (ET) to maintain “high-health” status of swine populations.

FIG. 13: Schematic representation of the use of marker-assisted selection (MAS) and marker-assisted allocation (MAA).

FIG. 14: Schematic representation of MATE® Phase I, “strategic mating”.

FIG. 15: Schematic representation of MATE® Phase II, “direct delivery” solution.

FIG. 16: Schematic representation of MATE® Phase II, “immuno-competent” solution.

### DESCRIPTION OF ILLUSTRATIVE EMBODIMENTS

The instantly disclosed invention sets forth a method for the rapid improvement of an animal population, based on phenotypic and/or genotypic information. Thus, using the instantly disclosed invention, one of ordinary skill in the art will be able to use newly described genetic or phenotypic information in order to rapidly modify swine herds by means such as either introducing a desirable trait and/or allele or to increases its frequency. This phenotypic/genotypic information may be obtained from a variety of sources. Such sources include, but not limited to, new genomic information and new biometric techniques.

Additional benefits provided by the various embodiments of the instant invention include a minimization of genetic lag time by maintaining the mean estimated breeding value (EBV) of the target herd at or above the mean EBV of the GN herd. Also, through the use of one or a small number of elite sires, the instant invention provides an efficient mechanism for making rapid shifts in gene frequencies in the target herd while maintaining a constant long term selection goal and protecting the GN herd against inbreeding. This is possible because according to various aspects of the current invention the elite sire(s) used in the target herd or herds are always selected from outside the target herd (i.e. they are from the GN herd). Therefore, the elite sires chosen will typically not be closely related to the females present in the target herd. An added benefit of the use of elite sires is that large numbers of half-sib progeny are produced that can be used to further reduce genetic variability, if desired. In summary, the invention provides maximum flexibility for making short-term changes in the target herd (as demanded by the customer(s)), while maintaining focus on long term selection objectives in the GN herd.

### Defined Terms

The following definitions are provided herein in order to aid the artisan of ordinary skill in more easily and fully appreciating the instant invention. As suggested in the definitions provided below, the definitions provided are not intended to be exclusive, unless so indicated. Rather, they are provided as preferred definitions, provided to focus the skilled artisan on various illustrative embodiments of the invention.

As used herein the term “acceptable rate of inbreeding” preferably means a level of inbreeding where the benefits of inbreeding outweigh any negative effects. In general inbreeding will accumulate in a “genetic nucleus” (GN) herd as a result of intra-herd selection. Typically, there is an inverse relationship between rate of inbreeding (ΔF) and rate of genetic progress (ΔG) in the short term. The optimum ΔF is the rate at which inbreeding is allowed to accumulate in order to optimize both short-term and long-term genetic gains. Under standard practice it is typically desired that ΔF be held to less than 1% per year. Methods to approximate ΔF are given, infra, in the “Illustrative Embodiments” section.

As used herein the term “allele” refers to a particular version or variant of a specified gene.

As used herein the term “BLUP” (which is an acronym for best linear unbiased prediction) refers to a statistical methodology introduced by Henderson (1959, 1963) that has become an animal breeding industry standard for predicting breeding values for individual animals.

With standard post-graduate training in animal breeding techniques, BLUP can be performed, by those of ordinary skill in the art, using any of the various commercially available computer programs that are used for genetic evaluation of an animal and/or herd. Most currently available programs are customized programs designed specifically to meet the needs of the breeding company. However, some standard software packages that are publicly available can be used to perform BLUP (e.g. “MTDF-REML” from Curt Van Tassell (curtvt@aipl.arsusda.gov); “PEST” from Eildert Groeneveld (eg@tzv.fal.de); “DMU” from Just Jensen (lofjust@vm.uni-c.dk); “MATVEC” from Steve Kachman (http://statistics.unl.edu/faculty/steve/software/matvec/); and “BLUPF90” from Ignacy Misztal (http://nce.ads.uga.edu/˜ignacy/newprograms.html)). Typical input parameters for BLUP programs include genetic and phenotypic parameter estimates, phenotypes, pedigrees, and fixed effects. BLUP models can be described most easily in matrix notation as follows:

y=Xβ+Zα+e,

where, y is the vector of phenotypic observations; β is a vector of fixed effects; X is an incidence matrix relating β to y; α is a vector of animal effects with a mean of zero and a variance-covariance matrix Gₐ; Z is an incidence matrix relating α to y; and e is a vector of residual effects with variance-covariance matrix R. Gₐ can be modeled as Gₐ=Aσ²ₐ, where A is the additive relationship coefficient matrix between animals, and σ²ₐ is the additive genetic variance. One of the requirements to obtain BLUP is to obtain the inverse of Gₐ, which can be computed very efficiently even with extremely large data sets (Henderson, 1976; Quaas et. al., 1984; Quaas, 1988).

As used herein the term “breeding plan” preferably refers to a program for improving herd genetics using the semen of a single elite sire (or a relatively small number of elite sires) that is sustainable over a prolonged period of time. That is a program that can be maintained consistently for weeks, months, and years. This is to be contrasted with the use of semen in a traditional plan that is not sustainable. That is a plan where the boar cannot consistently produce enough semen to maintain the “plan” over an extended period of time. As a hypothetical example, suppose that given a sufficient refractory period a boar might be able produce enough semen to inseminate 100 breeding females in a week's time. However, if the boar is unable to provide enough semen to maintain this level over an extended period of time (say that after providing enough semen to inseminate 100 breeding females in week one the boar only produces enough semen to inseminate 70 sows in week two); then inseminating 100 breeding females would not be part of a breeding plan, because it is not sustainable.

As used herein the term “breeding value” preferably refers to the expected value of an animal as a parent. It is a measure of the animal's expected progeny performance as compared with the mean of the population (see, Van Vleck, p. 186).

As used here in the term “correlative number” means a proportional number. For example, if each breeding female is inseminated three times, using three aliquots of semen, and 75 aliquots of semen are produced by a boar; then the correlative number of breeding females per boar would be 25.

As used herein the acronym “DIUI” denotes the phrase “deep intrauterine insemination.” This preferably refers to a method of artificial insemination wherein the semen is deposited within one or both of the horns of the uterus.

As used herein the term “economic trait locus” (ETL) preferably refers to a location on a chromosome that is linked to a “qualitative trait” providing economic value.

As used herein the term “effective population size” is a general term. Typically, “effective population size” is inversely related to the rate of inbreeding. It is calculated as a function of the number of males and females used as parents, for each generation, in the genetic nucleus herd.

As used herein the terms “efficient growth traits” and/or “performance traits” preferably refers to a group of traits that are related to growth rate and/or body composition of the animal. Examples of such traits include, but are not limited to: average daily gain, average daily feed intake, feed efficiency, back fat thickness, loin muscle area, and lean percentage.

As used herein the terms “elite sire,” “elite boar,” and “selected sire” can be used interchangeably. These terms preferably refer to the boar used to sire progeny that includes terminal boars. The “elite sires” are boars produced in the genetic nucleus (GN) herd that have extremely high (favorable) breeding value (i.e. superior genetic potential relative to the mean of the genetic nucleus herd population having “selected germplasm”). According to one aspect of the instant invention, an elite sire is used for breeding purposes in a target herd to produce progeny that includes terminal boars.

As used herein the preferred meaning for the term “embryo transfer” (ET) is the harvesting of fertilized oocytes(s) or embryo(s) from one female (embryo donor) and transfer of those embryo(s) into another female (embryo recipient) whose reproductive status is synchronized with that of the donor.

As used herein the preferred meaning for the term “in vitro fertilization” (IVF) is the harvesting of unfertilized oocytes(s) and the subsequent fertilization of those oocytes with semen in vitro (i.e. in the laboratory) instead of in vivo (i.e. in the live animal) as in standard ET. The fertilized oocytes(s) or embryo(s) from the oocyte donor are then transferred into another female (embryo recipient) similar to standard ET.

As used herein the term “estimated breeding value” (EBV) preferably refers to a specific numeric value for an animal that predicts its “breeding value”. EBV is often calculated using commercially available analysis programs (the output from BLUP and marker assisted BLUP programs are examples of EBV's).

As used herein the term “fixing a genotype” preferably means producing a population of pigs that are all homozygous for the same form of a particular gene or marker and thus have no genetic variability for that gene or marker.

As used herein the term “gene” refers to a sequence of DNA responsible for encoding the instructions for making a specific protein within a cell (including when, where, and in what abundance the protein is expressed).

As used here in the terms “genetic lag” and “negative genetic lag” refer to the genetic distance between the genetic nucleus (GN) herd and the target herd. Genetic lag is measured as the difference in the average estimated breeding values of the two herds at the same point in time. Traditionally we expect the target herd to have a lower genetic level than the GN herd and this difference is referred to as genetic lag. With the approach described in this disclosure the average genetic merit of the target herd is greater than the GN herd. This is what we termed “negative genetic lag”. It is not a common term.

As used herein the terms “genetic nucleus” (GN) and “genetic nucleus herd” preferably refer to the population that serves as the source of genetic improvement over time. Top ranking young males and females are identified from this population each generation and used back in the GN herd to replace older, lower ranking animals thereby creating genetic improvement that accumulates from one generation to the next.

As used herein the term “half-sib” refers to a group of animals all sharing one parent. Specifically, the term is most frequently used to refer to offspring sharing the same sire.

As used herein the term “health traits” preferably includes any traits that improve the health of the animal and/or herd. These include, but are not limited to: the absence of undesirable physical abnormalities or defects (like scrotal ruptures), improvement of feet and leg soundness, resistance to specific diseases or disease organisms, or general resistance to pathogens.

As used herein the term “high health” is meant to refer to a herd or population of animals that is characterized by the absence of certain diseases. It is a relative term. For example, in one embodiment of the instant invention, “high health” refers to the target herd from which terminal boars are sold. Such boars are free of porcine respiratory and reproductive syndrome (PRRS) and Mycoplasma pneumonia. The genetic nucleus (GN) herds may not be free of these two diseases.

As used herein the term “improved germplasm” preferably refers to change in the genome, improved frequency of genetic markers, genes, alleles of markers or genes, or any combinations of multiple markers or genes that is preferred over other forms of the genome that exist in the population. This includes forms of the genome that result in improved breeding values, but for which genotypes are not known. The term may, depending on the context, be used to refer to the genetic makeup of either a single animal or to the genetics of a herd, considered as a whole. Thus, the term “improved germplasm” covers both the introduction of a preferred trait in an individual and an increase in frequency of expression of a desired allele within a herd.

As used herein the term “level of selected germplasm” preferably refers to the frequency of animals in the herd having the “selected germplasm” or the degree to which breeding values have improved as a result of the selected germplasm.

As used herein the term “locus” refers to a specific location on a chromosome (e.g. where a gene or marker is located). “Loci” is the plural of locus.

As used herein the term “MA-BLUP” (an acronym for marker-assisted BLUP) is a method of analysis that utilizes the same inputs as BLUP (see above) and additionally adds the animal's marker genotype to the calculus. As with BLUP, MA-BLUP models can be described most easily in matrix notation as follows:

y=Xβ+ZKν+Zμ+e

where, y is the vector of phenotypic observations; β is a vector of fixed effects; X is an incidence matrix relating β to y; ν is the vector of additive effects at the marked QTL with a mean of zero and a variance-covariance matrix G_{ν}, and μ is the vector of additive effects of the remaining unmarked QTL with mean of zero and variance-covariance matrix G_{μ} (i.e. animals effects, previously represented by α, are subdivided into ν and μ, as a=Kν+μ, where K is the incidence matrix relating ν to α). Z are incidence matrices relating Kν and μ to y; e is a vector of residual effects with variance-covariance matrix R. To perform MA-BLUP, inverses of G_{ν} and G_{μ} need to be calculated. The inverse G_{μ} can be obtained as with G_{α} in regular BLUP (see above). The inverse for G_{ν} can be computed efficiently for large data sets where marker genotypes can be inferred (Fernando and Grossman, 1989), and in the case where marker genotypes are not known (Hoeschele, 1993; van Arendonk et al., 1994; Wang et al., 1991; Wang, et al., 1995). Additional information on how to perform MA-BLUP is also available (Wang et al., 1998; Totir, 2002).

As used herein the term “marker” refers to a sequence of DNA that has a specific location on a chromosome that can be measured in a laboratory. To be useful, a marker needs to have two or more alleles. Common types of markers include, but are not limited to: RFLP=restriction fragment length polymorphism; SSR=simple sequence repeat (a.k.a. “microsatellite” markers); and SNP=single nucleotide polymorphism. Markers can be either direct, that is, located within the gene or locus of interest, or indirect, that is closely linked with the gene or locus of interest (presumably due to a location which is proximate to, but not inside the gene or locus of interest).

As used herein the preferred meaning for the term “marker assisted allocation” (MAA) is the use of phenotypic and genotypic information to identify animals with superior estimated breeding values (EBVs) and the further allocation of those animals to a specific use designed to improve the genetic merit of terminal boars for sale or to improve the genetic nucleus or target herds.

As used herein the preferred meaning for term “marker-assisted embryo transfer” (MATE®) is process that allows a breeding company to simultaneously improve the health and genetic level of germplasm provided to customers. The process uses marker-assisted selection (MAS) as well as embryo transfer (ET) and/or in vitro fertilization (IVF) to produce and select breeding stock, transfer germplasm to customers and further select the resulting offspring in the customer's environment. Thus, by combining MAS and ET/IVF, MATE® provide a synergy and level of improvement not heretofore attained by the separate use of these two technologies.

As used herein the preferred meaning for the term “marker-assisted selection” (MAS) is the use of phenotypic and genotypic information to identify animals with superior estimated breeding values (EBVs) for selection and use as breeding animals for the genetic nucleus GN herd.

As used herein the term “meat quality trait” preferably means any of a group of traits that are related to the eating quality (or palatability) of pork. Examples of such traits include, but are not limited to muscle pH, purge loss, muscle color, firmness and marbling scores, intramuscular fat percentage, and tenderness.

As used herein the term “polymorphism” refers to the variation that exists in the DNA sequence for a specific marker or gene. That is, in order for a polymorphism to exist there must be more than one allele for a gene or marker.

As used herein the term “production nucleus” (PN) herd refers to a type of target herd, specifically a target herd designated, predominately, for the production of terminal boars for sale. The genetic merit of the terminal boars is primarily determined by the “elite sires” that are selected for use in the PN herd and secondarily by the selection of female progeny of the elite sires to be used as breeding animals in the PN herd.

As used herein a “qualitative trait” is one that has a small number of discreet categories of phenotypes and for which the genetic component is generally controlled by a small number of genes (“qualitative trait loci”).

As used herein the term “quantitative trait” is used to denote a trait that is controlled by several genes each of small to moderate effect. The observations on quantitative traits often follow a normal distribution.

As used herein the term “quantitative trait locus (QTL)” is used to describe a locus that contains polymorphism that has an effect on a quantitative trait.

As used herein, a “target herd” is a non-GN herd of female swine to which elite boars from the GN herd are mated for the purpose of transferring desirable genes/traits from the GN to the target herd.

As used herein the term “reproduction trait” refers to any of a group of traits that are related to swine reproduction and sow productivity. Examples include, but are not limited to, number of piglets born per litter, piglet birth weight, piglet survival rate, pigs weaned per litter, litter weaning weight, age at puberty, farrowing rate, days to estrus, and semen quality.

As used herein the term “selected germplasm” preferably refers to any form of the genome, including genetic markers, genes, alleles of markers or genes, or any combinations of multiple markers or genes that is preferred over other forms of the genome that exist in the population. This includes forms of the genome that result in improved breeding values, but for which genotypes are not known.

As used herein the term “specific pathogen-free” (SPF) preferably refers to the removal of a specific pathogen from a particular environment by any or all means possible; such that no measurable existence of the pathogen can be found.

As used herein the term “swine production herd” or “production herd” refers to a collection of animals whose primary purpose is to produce pigs that will be shipped to market for meat purposes.

The terms “terminal boar” and “terminal sire” are used, herein, interchangeably and preferably refer to a boar that is used to sire progeny that are harvested for pork. This is one type of animal produced by an embodiment of the instant method and will typically be sold to pork producers.

The term “terminal dam” refers to a female pig used in a swine production herd to provide the offspring, which are raised and sold to market (whether for meat or other products).

As used herein the term “useful life” for a boar preferably refers to the period of time during which it would be desirable to retain a particular boar for breeding purposes (specifically, the period during which use of the boar's semen will likely result in genetic improvement of the herd). Various factors must be taken into consideration when determining the “useful life” for the boar. These factors include, but are not limited to semen quality, the period of time when the boar's female offspring are mature for breeding (breeding the boar to his daughters often results in undesirable inbreeding), the availability of superior boars.

### ILLUSTRATIVE EMBODIMENTS

As noted above various embodiments of the instant invention are directed to methods for rapidly providing improved swine genetics. The instant invention encompasses improvement in swine genetics, both at the level of the individual animal (e.g. to provide terminal sires or dams for use in a swine production herd) and at the herd level (which would allow for the rapid introduction of a preferred trait or the rapid elimination of an undesirable trait).

One particular embodiment of the instant invention provides a method for producing terminal swine parents (i.e. terminal sires and terminal dams) and/or replacement animals (for the genetic nucleus and/or target herds). In one aspect of this embodiment the method comprises the steps of: a) selecting a trait or traits for which improvement is desired; b) identifying an elite sire which possesses germplasm which provides the means for improving the selected trait or traits; c) using semen aliquots from the selected elite sire which to breed a correlative number of females in a target herd; d) thereby producing offspring from the females bred with the elite sire's semen; and e) selecting the offspring exhibiting improved germplasm for use as terminal parents or replacement animals.

As was noted, supra, the instant invention provides methods that allow swine breeders to use a very few or, if desired, even a single boar to inseminate and/or impregnate an entire generation: of females in a target herd. This makes it possible to produce rapid changes in the allelic frequencies of one or more alleles in the target herd without jeopardizing long-term breeding objectives in the GN herd.

To expand upon this concept, one embodiment of the instant invention is particularly useful when there is a need or desire to establish multiple target herds of different sizes and having different selection objectives. According to one aspect of this embodiment, separate elite sires can be identified in the same GN herd and selected for use, according to the needs of marketplace. Each separate elite sire can be chosen based on its possession of a specific trait or quality. Subsequently, each individual elite sire can be used to sire progeny in a specified target herd. Swine production according to this method would provide several differentiated products, all produced from the same GN source. This embodiment of the invention illustrates both the power and flexibility of the invention to create multiple new and if desired highly specialized products while concomitantly maintaining a sound long-term selection program at the GN level.

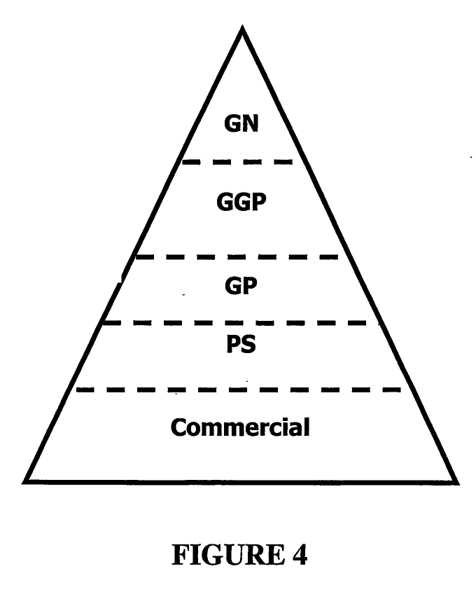

Various aspects of this embodiment of the invention can be further extended to provide significant improvements for the maternal side of a breeding program. Accepted practice in the swine industry is to use a multi-generational breeding program (pyramid, see FIG. 4) to produce crossbred females that are a combination of two or more purebred GN lines. At the top of the pyramid one maternal purebred GN line is used as the base sow population that is mated to boars of another maternal genetic line to produce crossbred females. The crossbred females may in turn be mated to boars of a third maternal genetic line to produce a particular female product that is ultimately used to raise market hogs.

Those skilled in the art refer to the various generations of swine based on their relationship to the terminal market hogs. For instance, the parents of the terminal market hog are referred to as a parent males or parent females; the next generation removed is referred to as grandparent males and females; etc. In order to reduce risk of disease from the transport of livestock from one site to another, swine producers may maintain and produce internally one or more generations on the maternal side of the breeding program. In some cases the swine producer will maintain the entire pyramid internally, including the purebred base population (target herd). This target herd is commonly mated with semen from boars in the breeding stock supplier's GN herd. According to one embodiment of the instant invention a single elite sire from the GN is used to mate all females in the target herd for an entire generation and the elite sire is changed from one generation to the next.

As noted, supra, a “target herd” is a non-GN herd of female swine to which elite boars from the GN herd are mated for the purpose of transferring desirable genes/traits from the GN to the target herd. According to various embodiments of the instant invention, a primary purpose of the described process is the eventual genetic improvement of commercial swine products. According to various embodiments of the instant invention, the commercial swine products may be direct progeny of animals in the target herd or may be descendents of animals in the target herd. The target herd may be made up of purebred females from the same genetic line as that of the GN herd or may be made up of any swine females for which it would be desirable to include genes/traits from the GN herd.

According to one aspect of this embodiment of the invention the target herd is for producing purebred terminal boars. This target herd may also be referred to herein as a “boar multiplier herd” or a “production nucleus herd”. Typically, production nucleus herd is a purebred herd that is of the same genetic line as the GN herd and is mated to elite sires from the GN herd for the purpose of transferring genes/traits to the terminal boar product. This transfer is accomplished through at least two means: 1) genetic transfer from the elite sires to their terminal boar progeny; and 2) genetic transfer from the elite sires to their female progeny that are selected back into the target herd, as replacement animals, and thereby contribute to future generations of terminal boars.

According to another aspect of this embodiment of the invention the target herd is a “target herd for producing crossbred terminal boars”. This target herd may also be referred to as a “crossbred boar multiplier herd”. In this aspect of the current embodiment the target herd is not the same genetic line as the GN herd. The target herd is mated to elite sires from the GN herd for the purpose of transferring genes/traits to the terminal boar product. The transfer is accomplished through genetic transfer from the elite sires to their terminal boar progeny. Female offspring of the elite sires are not used as breeding stock and do not contribute to future generations of terminal boars.

According to another aspect of this embodiment of the instant invention the target herd is a “target herd for producing purebred gilts.” This herd may also be referred to as an “external GN herd” or a “production nucleus herd” or a “daughter nucleus herd”. This is a purebred herd that is the same genetic line as the GN herd and that is mated to elite sires from the GN herd for the purpose of transferring genes/traits to the parent gilt product. The transfer is accomplished through genetic transfer from the elite sires to their purebred gilt progeny that in turn become ancestors of parent gilts through successive generations of crossing to boars of other maternal genetic lines or through using them as replacements in the target herd.

According to another aspect of this embodiment of the instant invention the target herd is a “target herd for producing crossbred gilts.” This herd may also be referred to as a “GGP herd” (great grandparent herd), “GP herd” (grand parent herd), “crossing farm” herd, or a “crossbred gilt multiplier” herd. This target herd is not the same genetic line as the GN herd and it is mated to elite sires from the GN herd for the purpose of transferring genes/traits to the parent gilt product. The transfer is accomplished through genetic transfer from the elite sires to their crossbred gilt progeny that are either parent gilts or will become ancestors of parent gilts through successive generations of crossing to boars of other maternal genetic lines.

In certain aspects of this embodiment of the invention the semen from one or more terminal sires is used to breed all or substantially all of the females in the target herd. In a preferred aspect of this embodiment, the semen from a single elite sire is used to breed all or substantially all of the females in the target herd.

In aspects of these embodiments of the invention the semen of the elite sire is used in accordance with a “breeding plan” wherein the semen aliquots are used to inseminate a sufficient number of females in order to produce an average of 160 half-sib offspring per week. More preferably, the plan is carried out so as to produce an average of at least 250, 320, 400, 500, 640, 700, 800, 900, 1000, 1100, or 1280 half-sib offspring per week.

According to one aspect of this embodiment of the invention the elite sire is selected from a genetic nucleus herd. However, the elite sire may be selected from any suitable source, so long as it is an exceptional example of the trait(s) or attribute(s) which have been selected for improvement.

Elite sires may also be produced from the GN or other source resulting from specialized matings among selected parents that have the special trait, genotype, attribute or extremely superior EBVs. Boars from the resulting litters may then be genotyped and/or phenotyped to determine which received the desired genetic traits from the parents. In the particular case where the GN or other source of elite sires has a lower health level than the target herd, embryo transfer (ET) with embryo washing for pathogens may be used to produce elite sires from selected parents in order obtain the elite sires without bringing in undesirable pathogens.

In various aspects of the invention the breeding plan is designed such that it provides for a sustainable production of offspring. For example, if a breeding plan is defined as providing an average of at least 160 half-sib offspring per week from an elite sire. This means that the breeding plan must be carried out such that it is practicable to produce 160 offspring from a single sire, week after week for an extended period of time. Specifically the breeding must be carried out so that the elite sire is capable of producing sufficient semen to average this number of offspring every week for periods of from one week to two years or more.

In one aspect of this embodiment the breeding plan is capable of being sustained for at least the maximum useful life of the sire. The maximum useful life of the sire is determined by a number of factors, including but not limited to: the time before which a superior elite sire becomes available, the time before his daughters have attained breeding age (this limitation is important to prevent undesirable inbreeding), until he is injured or until his semen production is of insufficient quality and/or volume to be useful, or until the breeding goal for the herd changes and the sire in question no longer ranks highly.

The actual length of time the breeding plan is carried out is not, necessarily critical. What is important is that the program is carried out for a length of time sufficient to meet the goals of the plan. It is important, however, that the plan be carried out in such a manner that would be possible to maintain the offspring production rate for an extended period of time (e.g. one to two years, or more), if desired.

This does not mean that to fall within the scope of the present invention such a number of offspring must necessarily be produced every week, it means only that the plan is designed such that the elite sire would be capable providing sufficient semen to sustain this level of production (see the definition of “breeding plan,” supra). Various embodiments of the instant invention provide for ways of sustaining such a high level of offspring production.

The average boar ejaculate contains 70 billion spermatozoa. It has been estimated that an average boar used for artificial insemination (Al) has its semen collected about 1.1 times per week (Watson et al.). It has also been estimated that, employing the current, widely used AI methods it requires 2.2 inseminations at a dose of about 3 billion spermatozoa per dose to service a breeding female. Thus, under current practices an average boar only produces enough sperm to service about 12 females per week ((70×1.1)/(2.2×3)) resulting in the production of about 100 half-sib progeny per week (M. T. See). Therefore, in order to achieve the productive levels required by the instantly described invention, it is necessary to employ methods which allow for improved production from the same amount of spermatozoa.

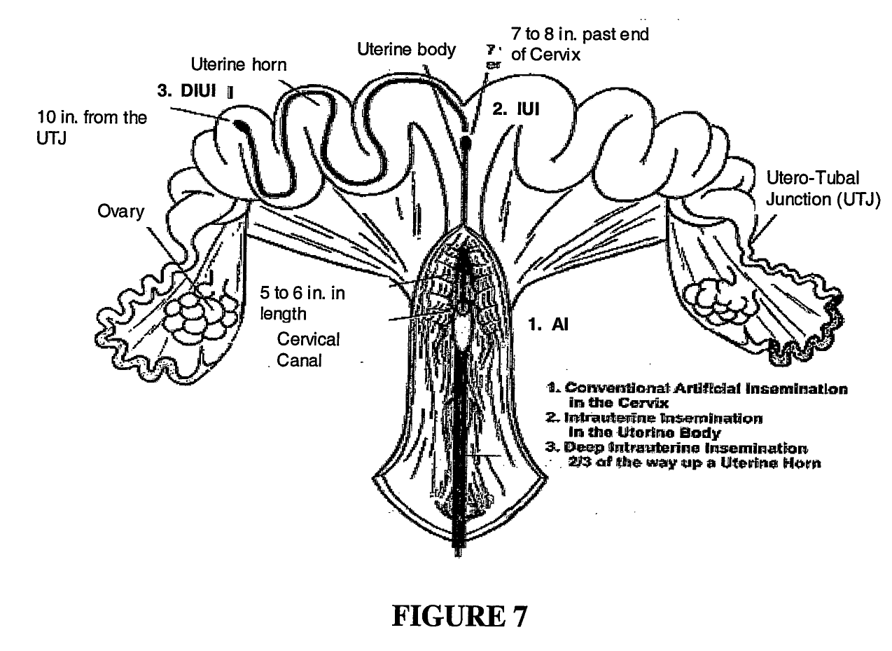

According to one aspect of this embodiment, the high level of offspring production is achieved and maintained through the use of deep intrauterine insemination (DIUI). DIUI requires far fewer spermatozoa to achieve the same result as conventional AI or even IUI because, rather than being deposited outside of the cervix (as in conventional AI) or just interior, but proximal to the cervix (as in IUI), the semen is deposited deep in the uterine horn (much closer to the junction of the uterus and the fallopian tube) see FIG. 7 and the discussion infra.

### Artificial Insemination

There are three currently available ways to inseminate a sow, namely traditional artificial insemination (AI), intrauterine insemination (IUI), and deep intrauterine insemination (DIUI). Traditional AI has been around since 1982, though it has only been widely used since 1996 (currently 85% of breeding in the industry is accomplished by traditional AI). In contrast, IUI and DIUI were developed and put into use only since about 1998. Each of these three methodologies is described below. Additionally, FIG. 7 shows the reproductive tract of a female pig and the typical locations where semen is deposited for each of the three listed methods of artificial insemination.

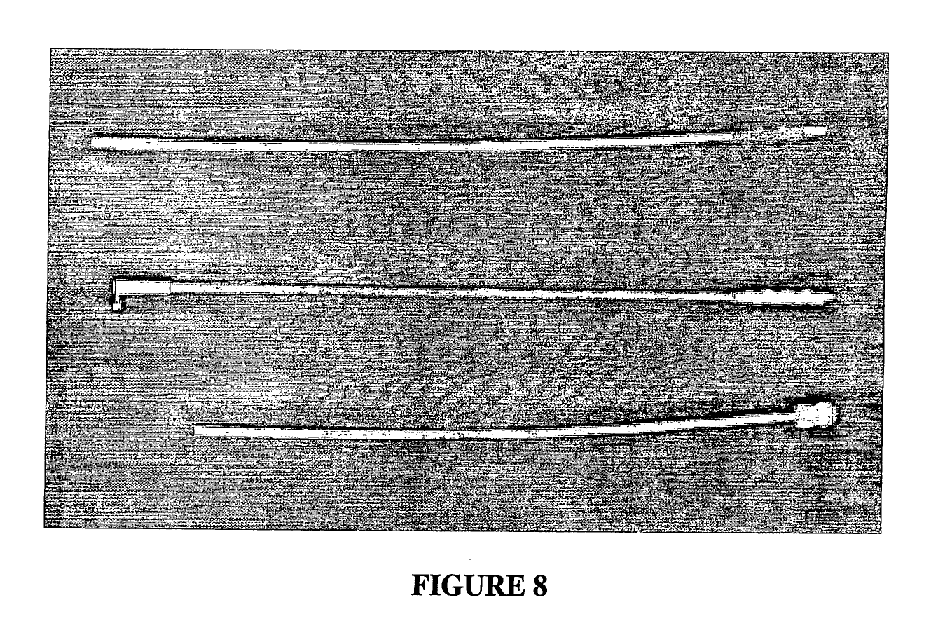

Traditional artificial insemination utilizes what is considered a standard AI cervical catheter and deposits semen directly in the cervix. A standard AI cervical catheter generally measures 56 cm long and is composed of a hard plastic material with foam tipped end (a picture showing examples of this type of catheter is provided as FIG. 8). Typically traditional AI is carried out using semen aliquots containing from about 2.5 to 4 billion spermatozoa per dose. The semen is often diluted using a “semen extender” and delivered in a volume of from about 70 ml to 10 ml per dose.

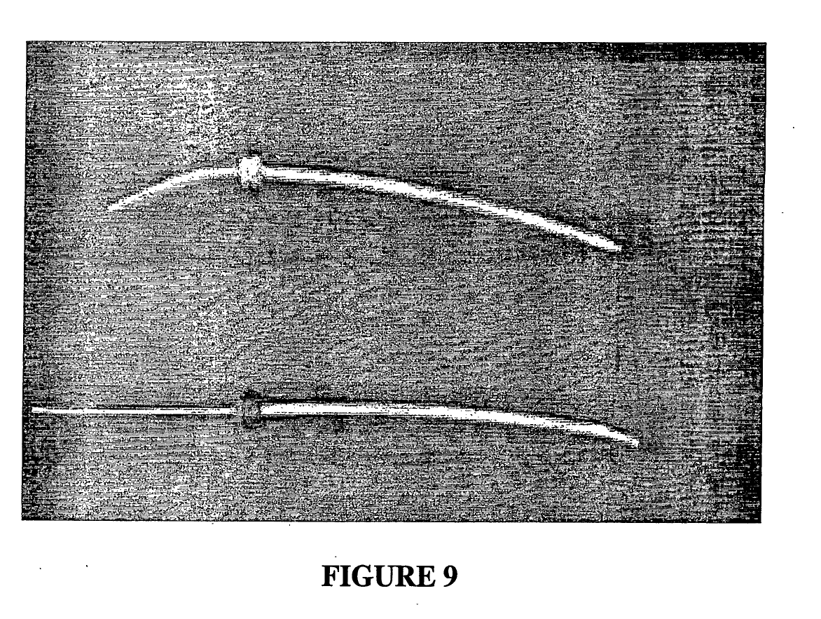

As shown in FIG. 7, intrauterine insemination is carried out by depositing the semen aliquot in the uterine body (typically about 7 to 8 inches from the cervix. IUI is carried out using intra-uterine insemination catheters that are generally about 70 to 80 cm long. The semen is deposited into the uterine body in aliquots of about 1-1.5×10⁹ spermatozoa per dose. As with standard AI the semen is normally “extended” and is delivered in a volume of about 40 ml to 80 ml per dose. The catheters used usually comprise a standard AI cervical catheter with an inner soft pliable catheter that will traverse the cervix and penetrate into the uterine body. Examples of this type of catheter are shown below in FIG. 9.

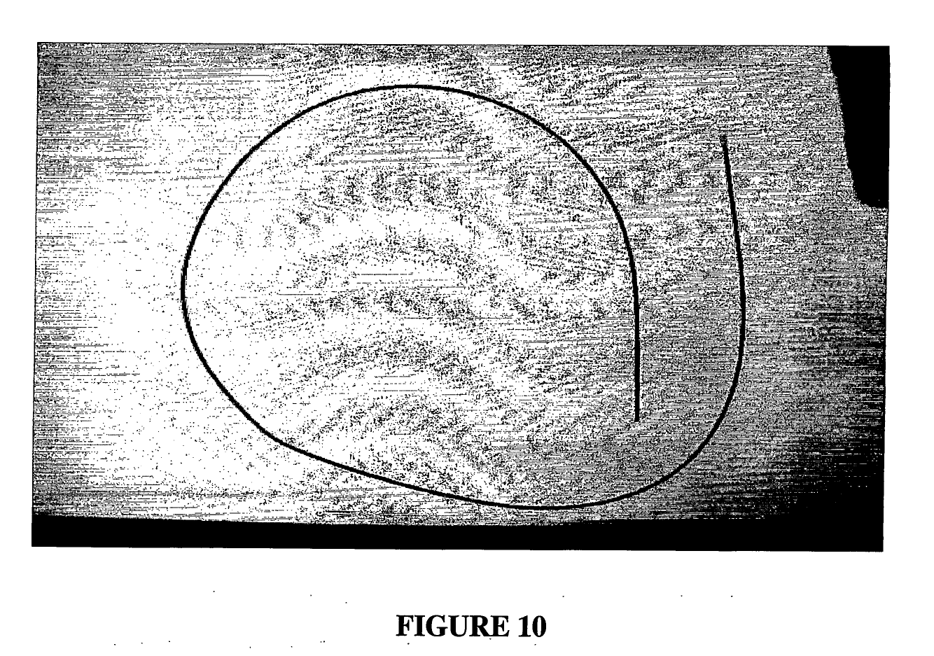

As indicated in FIG. 7, DIUI is usually performed by depositing the semen in the upper (anterior) ⅓ of the uterine horn. Typically DIUI is done with semen aliquots containing from 50-900×10⁶ spermatozoa per dose. Again the semen is “extended” and delivered in a volumes of about 5 ml to about 20 ml per dose. DIUI is performed using a device comprising a standard AI catheter and a DIUI catheter (the DIUI catheter is usually about 150 cm long and is constructed of a soft pliable outer material and having a spring core to maintain the needed balance of flexibility and resistance). The standard AI cervical catheter is used to facilitate threading the DIUI through the cervix and up into the uterine horn. A photo of a DIUI catheter is shown as FIG. 10. Catheters and methods for carrying out DIUI are known to those skilled in the art. For example, see Garcia et al., U.S. Pat. App. Pub. No. US 2002/0072650 A1; Martinez et al., Reproduction 123:163-170, 2002; Martinez et al. Reprod. Supp. 58: 301-311, 2001; and Hazeleger, WIPO Pub. No. WO 99/27868, each of which is herein incorporated by reference.

Gilts and sows exhibit prolonged muscle contractions when they come into heat for example they exhibit what is known as a “standing reflex”. Despite these intense muscle contractions, in sows it is still possible to pass either an IUI or DIUI device through the cervix and conduct the artificial insemination. However, with gilts, the passage is typically too restricted to allow insertion to take place. Thus, in most cases in order to use IUI or DIUI with gilts it will require use of a means to relax the gilt prior to insemination (perhaps through the use of a muscle relaxant). Nevertheless, semen from the elite sire(s) can still be used to breed the gilts by traditional AI fashion. In practice this means that the breeding program must take this factor into account in planning for the most efficient use of the semen from the elite sire(s).

In a preferred aspect of this embodiment the semen is deposited in the anterior ½ of one or both uterine horn(s), that is closer to the utero-tubal junction (UTJ, i.e., the junction between the uterus and the fallopian tube) than to the cervix. In another aspect of this embodiment, the semen is deposited in the anterior ⅓ of one or both uterine horn(s). In yet another aspect of this embodiment of the invention, the semen is deposited at or substantially at the UTJ.

According to another embodiment of the invention, in order to maximize the effectiveness of this AI, the variable estrus cycle of the female is hormonally synchronized to ensure that the timing of the AI is optimized to maximize the efficiency of insemination. For example, a gonadotropin preparation (P.G. 600®, Intervet, Inc.) is currently available to induce porcine estrus synchronization.

Some studies have suggested that fertility comparable to that achieved with 3×10⁹ spermatozoa, using conventional AI techniques, may be achieved with as few as 10×10⁶ sperm using DIUI (especially where the sperm are deposited at or near the utero-tubal junction in each uterine horn of a synchronized breeding female) (see, M. T. See).

According to one embodiment of the instant invention the breeding plan comprises the use of DIUI performed using a device as described in patent publication no. US2002/0072650 A1 (Garcia et al.) which is herein incorporated by reference.

Researchers have employed related devices to achieve acceptable results using spermatozoa doses of from 10×10⁶ to 1×10⁹ spermatozoa delivered through the use of either a fiber optic endoscope or a secondary catheter inserted through a modified AI catheter (see, M. T. See, Table 1).

In various embodiments of the invention the AI is carried out by non-surgical or surgical techniques using doses of from 10×10⁶, or fewer spermatozoa to 1.5×10⁹ spermatozoa. In one aspect of this embodiment between 10 million and 1 billion spermatozoa are used. In another aspect, doses of from 10 million, 20 million, 30 million, 50 million, 75 million, 100 million, 150 million, or 250 million, or more spermatozoa are used.

According to one embodiment of the invention, either a single elite sire or, alternatively, a small number of sires is used to breed all, or substantially all, of the females in the target herd. As used herein “substantially all” preferably means all but a very small number or percentage of the females in the target herd. One of ordinary skill in the art will understand that there may be reasons for not breeding a certain, small percentage, of the target herd, whether for health reasons or for any other economic reason.

Failure to breed a small number of the target herd females, does not take one outside the scope of the instant invention. According to a preferred aspect of this embodiment all or substantially all of the target herd females are bred using the semen from a single elite sire.

Other methods that could also used and fall within the envisioned scope of the instant invention (that is methods that would sufficiently extend the productivity of a single elite boar). For example the use of in vitro fertilization and/or embryo transfer might be routinely used to maximize the number of females bred using the semen from a single boar.

In various aspects of the invention the animals in the herd to be improved are selected for the presence of desirable traits or markers, including but not limited to: the presence or absence of specific genes and/or alleles, health traits, reproduction traits, meat quality traits and efficient growth traits. Animals possessing the desirable traits/markers are then used according to the methods described herein to improve the herd.

One aspect of this embodiment of the invention anticipates providing terminal parents and or a target herd for modification or “fixing” of a single gene, allele, or locus. There are known instances where a single gene conveys a desirable or undesirable characteristic. Examples of this type of single locus gene include the Rendement Napole gene and the Halothane gene, both of which impact animal performance, carcass composition and pork quality.

Rendement Napole (RN) gene has an economically important impact on the meat quality traits of swine. The presence of the dominant allele, RN, is associated with inferior meat quality attributes. The causative mutation for the RN gene is currently unknown; however, a DNA test is used to classify animals as carriers (RN⁻/RN⁻ or RN⁻/rn⁺) or negative (rn⁺/rn⁺) for the RN gene.

The ryanodine receptor gene, which is also referred to as the “halothane gene,” impacts carcass lean percentage, meat quality, and litter size in pigs. The presence of two recessive alleles causes a stress susceptibility condition known as porcine stress syndrome (PSS) that is characterized by sudden death of pigs when they are physically stressed. This genotype has a beneficial effect on lean percentage, but this is accompanied by poor meat quality attributes and a condition known as pale, soft, exudative (PSE) pork, as well as smaller litter size in females. The “halothane” gene derives its name from the fact that pigs with the homozygous recessive genotype exhibit malignant hyperthermia on exposure to the gas halothane (2-bromo-2-chloro-1,1,1-trifluoroethane, an inhaled anesthesia). Exposure to halothane gas was once used to detect pigs with homozygous recessive genotype, but this has been replaced with a DNA test that allows for the identification of all three (homozygous dominant, heterozygous, and homozygous recessive) genotypes. Using this test, the dominant allele for the halothane gene has been fixed in many commercial populations.

Thus, various embodiments of the invention provide methods for modulating one or more traits selected from, but not limited to, the group comprising: a single gene, locus, or allele, an economic trait, an economic trait locus, a qualitative trait, a quantitative trait, an efficient growth trait, a meat quality trait, a reproduction trait, and a health trait.

In certain aspects of these embodiments a single allele may be either rapidly “fixed” in the population or, alternatively, rapidly eliminated from the population, depending on the specific design of the breeding plan. In this context, “fixing an allele” refers to increasing the frequency of a specific (presumably favorable) allele to 100% (or substantially 100%) in the target herd. The result is that all, or substantially all, members of the target herd will be homozygous for the (favorable) allele that has been “fixed.”

In other aspects of these embodiment of the invention the efficient growth trait is selected from, but is not limited to, the group comprising: average daily weight gain, average daily feed intake, feed efficiency, back fat thickness, loin muscle area, carcass lean percentage, and percentage of primal cuts.

In other aspects of this embodiment of the invention the meat quality trait is selected from, but is not limited to, the group comprising: muscle pH, purge loss, drip loss, muscle color, muscle firmness, marbling scores, intramuscular fat percentage, cooking loss, belly thickness, and meat tenderness.

In other aspects of this embodiment of the invention the reproductive trait is selected from, but not limited to, the group comprising: ovulation rate, embryo survival percentage, number of piglets born per litter, piglet birth weight, piglet survival rate, pigs weaned per litter, litter weaning weight, age at puberty (i.e. the age at which the female undergoes her first estrous cycle), farrowing rate, days to estrus, and semen quality.

In other aspects of this embodiment of the invention the health trait is selected from, but not limited to, the group consisting of: the absences of undesirable physical abnormalities or defects (e.g., a propensity for inguinal hernia (scrotal rupture), cryptorchidism, atresia ani, splay leg, Halothane gene, Rendement Napole (RN) gene, and etc.), improved feet or leg soundness, resistance to specific diseases or specific pathogenic organisms, and general resistances to pathogens. A more extensive list of genetically inherited diseases can be found in the Internet as Online Mendelian Inheritance in Animals (OMIA) at www.angis.org.au/Databases/BIRX/omia/.

Other embodiments of the instant invention provide methods of rapidly fixing an allele in a target herd. In preferred aspects of this embodiment the gene is “fixed” in four generations or less; in a more preferred aspect of this embodiment the allele is fixed in the herd population in two or three generations. It will be understood by those of skill in the art that rapid fixing an allele in a herd population may require eliminating from the herd those animals which do not possess the desired allele. Thus, these methods comprise providing a target herd, selecting a trait for which improvement is desired; breeding the females of the target herd using semen from an elite sire (from a GN herd), where said elite sire has the desired trait, producing offspring; and identifying those target herd animals, including female offspring that are either heterozygous or homozygous for the desired allele; retaining as breeding stock those animals which are heterozygous or homozygous for the desired allele; repeating the foregoing steps a sufficient number of times to “fix” the desired allele in the target herd.

Various embodiments of the presently disclosed invention are also directed to herds and individual animals produced by the methods described herein. This includes, but is not limited to production nucleus herds, crossbred boar multiplier herds, daughter nucleus herds, and gilt multiplier herds. As will be understood, the instant methods are effective for rapidly providing (in high numbers, if demanded) terminal parent animals having specific genetic traits desired by swine producers.

Animals may be selected for use according to the instant invention by any suitable means; for example using computer programs or other means for recording parentage/pedigree and selecting the most suitable pairings. The use of computer programs can be further enhanced with the input of biometric data, including the use of molecular genetic analyses.

Methods of molecular genetic analysis are well known to those of ordinary skill in the art and include, but are not limited to, DNA fingerprinting (which can be based on RFLPs=restriction fragment length polymorphisms; simple sequence repeat (SSR, a.k.a. “microsatellite” markers), polymerase chain reaction (PCR) amplified fragments, especially multiplexing PCR (the simultaneous amplification of several sequences in a single reaction)) and single nucleotide polymorphism (SNP, which analyzes single nucleotide differences in, for example, an allele for a gene of interest).

Various embodiments of the instant invention further provide for the use of the markers described supra. That is, the instant invention provides as one of its aspects, a means a means of using markers to identify those animals suitable for use in accordance with the invention. This process is termed MAS (marker assisted selection). The invention also envisions the use of MAA (marker assisted allocation). Through the use of MAA, selected animals are allocated for use so as to most effectively and efficiently bring about the desired genetic improvements in progeny animals.

In certain embodiments of the instant invention, information/data obtained from the analysis of various biometric measurements as well as other types of information (e.g., pedigree) can be weighted in a “selection index” in order to provide an evaluation of an animal's value as a parent, i.e., its estimated breeding value.

Estimated breeding values are affected (biased) by the herd and year or season in which the animal's performance is measured. In order to correct for this bias a procedure called BLUP (Best Linear Unbiased Prediction of breeding value) was developed (see, Animal Breeding, p. 84). There are currently several computer programs available from the authors of the software which can be used to calculate BLUP values. These include PEST (Dr. Eildart Groeneveld at E-mail eg@tzv.fal.deV), BLUPF90 and BLUPF90IOD (Dr. Ignacy Misztal, see nce.ads.uga.edu/˜ignacy/newprograms.html), and MTDFREML (Boldman, et al., 1995, see, aipl.arsusda.gov/curtvt/mtdfreml.html).

According to certain embodiments of the instant invention, the methods of the invention may be used to provide selected genetic improvement(s) at any particular level of swine production (e.g. at the level of the production swine (SP) herd, the genetic nucleus (GN) herd and/or the production nucleus (PN) herd). Alternatively, the instant methods may be used to provide concurrent genetic improvement at any combination of swine production level.

### Normal Limitations Due to Inbreeding Rate

In a traditional breeding strategy, changes in gene (allele) frequencies or trait emphasis are made relatively slowly over time since the selection changes are normally made only in the GN herd. One of the reasons that these changes happen relatively slowly is due to breeding procedures designed to control inbreeding rate.

Inbreeding is defined as the probability that two genes (i.e. alleles) at a locus are identical by descent (Malecot, 1948). The inbreeding level (F_{X}) (i.e. inbreeding coefficient) can be calculated from pedigree records tracing back to the founder animals of a given population as follows:

Fₓ=(½)a_{XsXd}

(where, a_{XsXd} is the additive genetic relationship between Xs and Xd; if X is the progeny of Xs and Xd)

Increased homozygosity due to inbreeding is generally perceived to have deleterious side affects such as inbreeding depression (i.e. a decrease in performance in production, reproduction, and fitness traits) and decreased genetic variation leading to reduced rates of genetic gain over time.

Inbreeding rate, ΔF, is defined as the increase in the inbreeding coefficient in one generation (Falcaner and Mackay, 1996), and can be approximated by:

ΔF=⅛Nₘ+⅛N_{f}

### Where, Nₘ and N_{f} are the numbers of males and females, respectively, contributing to the next generation.

As evident in this approximation, as fewer animals are selected as parents, inbreeding rate tends to increase. Unfortunately, increased selection pressure takes the form of selecting a smaller proportion of parents for the next generation. Therefore, swine breeding companies normally try to balance the extra genetic gain from selecting fewer parents against the resulting increase in inbreeding rate. Typically in swine populations, many females are selected to produce sufficient offspring for the next generation, therefore, inbreeding caused by female parents is not usually a concern. However, in order to limit the inbreeding rate and to maintain genetic variation in the herd, it is common practice to select more males than are strictly needed for reproduction purposes. This practice limits both the rate of genetic progress in the GN and the speed at which changes can be made in gene frequency and trait direction. When several sires must be selected as parents, it is difficult to find a set of sires that all have high breeding values with a particular genetic profile (e.g. specific genetic marker profile).

Other embodiments of the instant invention provide for GN herds, target herds, and/or SP herds which have been produced or modified through the use of the methods described herein.

According to one embodiment of the invention, the use of a target herd which is separate from the GN herd it is possible to separately improve the genetics of these two herds. In one aspect of this embodiment the GN herd contains the minimum number of females and males necessary to maintain high annual genetic gain with no or very low annual change in inbreeding. According to this aspect of the invention, the GN herd supplies one or a very few “elite sire” (also called “super sires”) for use in the target herd. Therefore, the target herd is not required to produce males as replacements for the next generation. As a result, as long as the current elite sire(s) selected are not closely related to the current females in the target herd, inbreeding levels in the resulting target herd offspring will not be a concern. Consequently, use of the instant invention has the advantage of allowing for a relatively intense selection pressure to be placed on desired traits in the target herd while concomitantly allowing for selection practices in the GN herd which minimize inbreeding (an maximize genetic variation) insofar as possible.

According to another embodiment of the invention the breeding plan is designed so that a “negative genetic lag” between the GN and the target herd is created. Whereas, typically, the time required to transfer germplasm from a GN to a target herd (e.g. PN) induces a so-called “genetic lag”, various embodiments of the current invention provide for a target herd wherein the offspring show negative genetic lag. That is, using embodiments of the current invention the best sire produced in the GN herd would be used in the target herd (e.g. elite sire) and would be better than the average of the top sires used in the GN herd, thereby, reversing the genetic lag (i.e. creating a “negative genetic lag”).

### Limitations Due to Multi-Trait Selection Indexes:

Typically, selection in a genetic nucleus herd is practiced via the use of a multi-trait selection index. In this approach, estimated breeding values are calculated for each economic trait for each animal based on pedigree and phenotypic information. The estimated breeding values are then weighted according to the relative economic value of each trait as well as the intended direction of selection for the population and incorporated into a single, multi-trait selection index. These multi-trait indexes incorporate several sources of information for each animal (e.g. phenotypic records on ancestors, progeny and the animal itself). Selection indexes determine the long-term genetic progress for the population and must be carefully constructed to balance needs of both the present and future marketplaces. Accordingly, if temporary changes in the market occur, a breeding company cannot justify completely changing the selection index to reflect those changes; especially if future market conditions are not likely to match the current, temporary conditions.

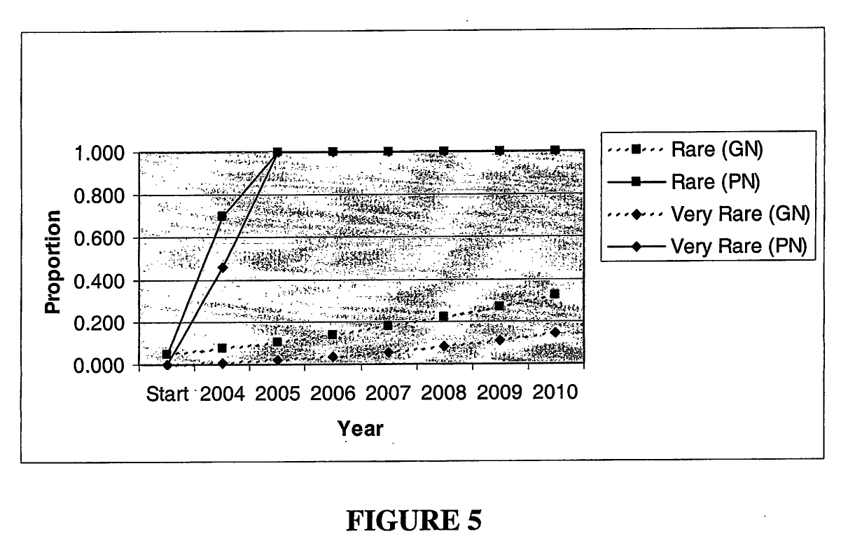

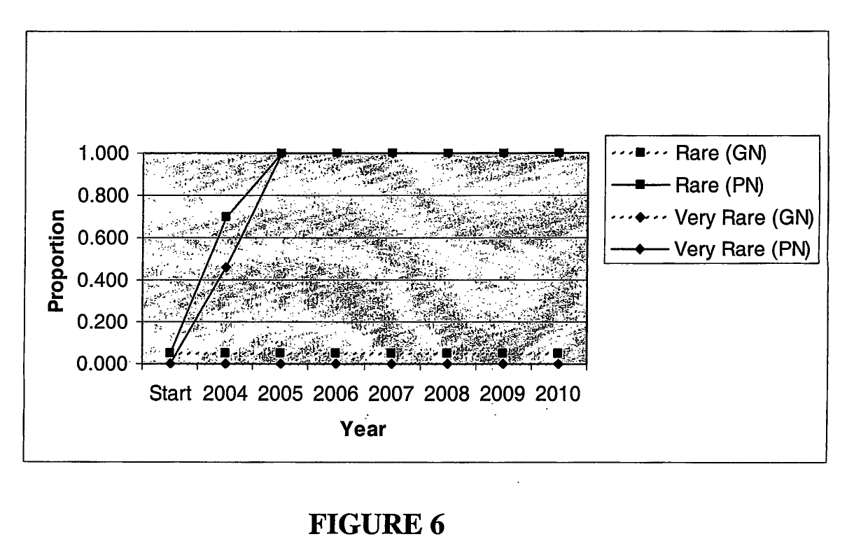

According to another embodiment of the invention, the target herd can change the direction of selection in a very short period of time relative to the GN. Consequently, if a temporary marketplace change takes place (e.g. a temporary switch to meat quality rather than growth efficiency) the elite sire(s) chosen can be the highest male in the GN for the temporary selection index. Similarly, if the marketplace demanded a specific marker profile, only elite sires matching this profile would be used. In a period of only two generations, the frequency of the desired allele could be increased to 100% or substantially 100%, even if the frequency of this allele was extremely low in the GN. As shown in FIGS. 5 and 6, selection for rare or very rare alleles could be increased much more rapidly in the target herd (PN) than in the GN due to much greater selection intensity that is possible.

FIG. 5 shows that the frequency of rare and very rare alleles can be increased in the target herd (PN) independently from the GN herd. This aspect of the current embodiment is particularly useful in situations in which selection for the rare allele is not favorable in the long term.

### Limitations Due to a Segmented Marketplace

The marketplace can often be segmented due to differing conditions and demands of individual customers. Typically the selection index in a GN herd is designed to meet the needs of the marketplace in general; consequently, it cannot focus specifically on individual customer needs. These limitations present a sub-optimal situation for delivery of germplasm to the customer. As described, various embodiments of the instant invention remedy this problem by allowing individualized selection based on the specific needs of the customer.

In another aspect of this embodiment the target herd contains the minimum number of females to meet breeding plan-specified market demands for target herd offspring. It is noted that it is possible, and in some instances desirable, to establish separate target herds in order to satisfy the demands of separate market segments. In this way, the breeding program can be regulated so as to produce just enough sires or dams to meet the demands of the various market segments.

### Two-Stage Selection

Typically, selection takes place on quantitative traits based on BLUP breeding values and ranked in a multiple-trait selection index. However, there are increasing numbers of economic trait loci (ETL) that have been discovered that have been reported to be associated with traits that are not normally considered in the multiple-trait selection index yet have a measurable economic value (e.g. health or meat quality traits).

A simple approach to use of these genes is through two-stage selection. In the first stage, animals could be genotyped for one or more ETL then pre-selected for the most favorable form (allele) of the ETL. Next, in the second stage, additional selection is performed on the remaining animals according to the traditional multi-trait selection index. This approach has the benefit of being relatively easy to apply and may reduce the number of animals for which regular phenotyping is necessary (e.g. gain on test, ultrasound measures of back fat and loin eye area, etc.).

Alternatively, the first stage can comprise a standard phenotyping procedures and rankings according to multi-trait BLUP EBVs. This is then followed by a second stage in which animals are differentiated according to their genotypes at one or more ETL. This second option does not present any savings in phenotyping, but could provide savings in genotyping if some animals rank too lowly to be considered for selection and therefore genotyping costs are not justified. In addition, some genotypes may have more value to certain customers than others and, therefore, marker-assisted allocation (MA) can be used to allocate specify animals to customers desiring a particular genotype. MAA can therefore be justified by charging a premium to customers receiving the specified genotype.

### Single-Stage (Multi-Trait Index) Selection

Simultaneously incorporating all available information at the time of selection, in the form of a single-stage multi-trait selection index, is the most efficient form of selection. Moreover this method results in the greatest long-term progress towards the stated breeding objective. Other selection strategies such as two-stage selection (above), tandem selection (i.e. alternating selection on different traits over multiple generations), or use of independent culling levels (i.e. eliminate animals not reaching a minimum culling threshold) have been shown to be less efficient than index selection (Van Vleck, et al., 1987). Nevertheless, these other methods are sometimes employed for reasons related to ease of use, cost or speed of implementation.

Index selection normally takes the form of a linear equation, as follows:

Hᵢ=ν₁A₁ᵢ+ν₂A₂ᵢ+ . . . +ν_{N}A_{Ni}

where, Hᵢ is the selection index value for animal i, ν₁, ν₂ and ν_{N} are the net economic values per unit of trait 1 through N, A₁ᵢ, A₂ᵢ and A_{Ni} are the additive genetic value for animal i for traits 1 through N. Additive genetic values for each trait can be calculated to include ETL information via MA-BLUP (described above). Further information is easily available regarding index selection (Van Vleck et al., 1987; Van Vleck, 1983).

One of the most difficult aspects of incorporating ETL information into multi-trait index selection is determining how to properly weight the new information relative to traditional trait phenotypic information. Since ETL information is often conditional on marker genotype information, this information can be difficult to include, because markers are not usually located directly at the ETL, but rather some distance from it. Recombination (chromosomal crossovers) can break down the linkage (strength of association) between the marker and the ETL, and tends to occur in proportion to the distance between the marker and the actual ETL. This recombination rate needs to be taken into account as well as situations where genotypes are not available on all animals.

This process has become much more feasible with the advent of MA-BLUP methodology (see above), whereby the ETL information is combined into the additive genetic breeding value for that trait for the animal. In the MA-BLUP scenario, marker information can be simultaneously included with phenotypic and pedigree information to predict breeding values. If the trait affected by the ETL is already included in the multi-trait selection index, then ranking and selection can proceed more or less as done previously.

However, if the ETL affects a new trait that is not currently in the breeding objective, then additional work must be done. First, to assess the economic value of the new trait and, second, to estimate the necessary genetic parameters surrounding the new trait (i.e. heritability, genetic variance and covariance with the other traits in the selection objective). Information regarding estimating genetic parameters and applications for BLUP models used in animal breeding is known to those of skill in the art (see, e.g. Henderson, 1984).

Accordingly, various embodiments of the instant invention use marker-assisted selection, marker-assisted allocation, embryo transfer and/or in vitro fertilization (discussed below) to further enhance the power and benefits of the instant invention.

### The MATE® Strategy

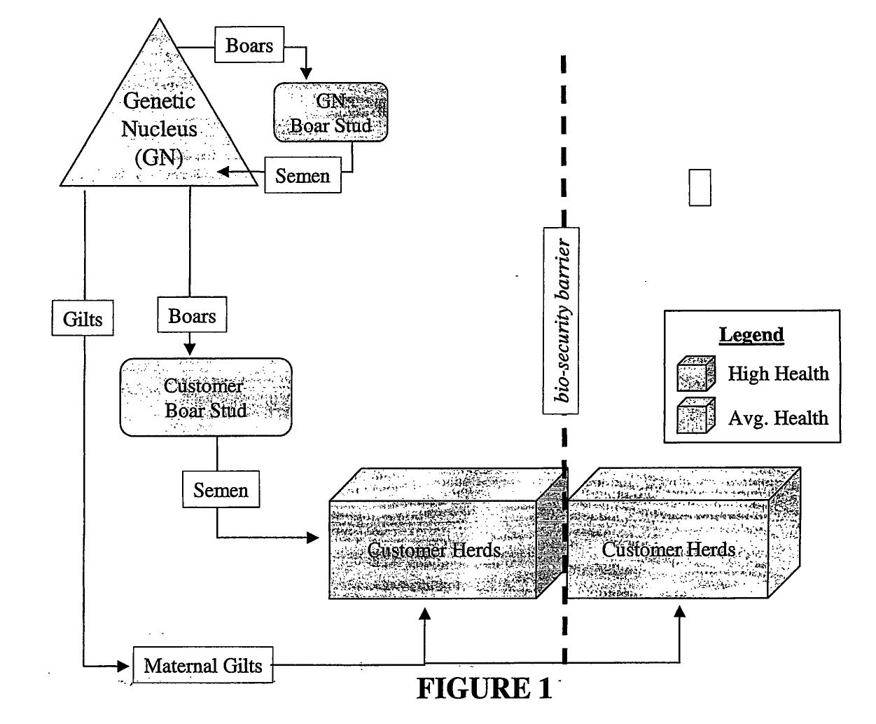

Due to concerns over bio-security in the animal agriculture industry, maintaining breeding stock populations in a pathogen-free environment is the current standard being employed by purveyors of farm animal germplasm (e.g. specific pathogen-free (SPF) swine nucleus herds (see FIG. 1) and SPF cattle artificial insemination (AI) centers). This methodology has the dual disadvantage in that it results in (a) additional maintenance and testing expenses; and (b) the requires the opportunity cost of lost genetic progress via culling of genetically superior, but infected, animals.

For example, when a pathogen is discovered in a swine nucleus herd, immediately the health status of the entire nucleus is assumed to be at risk. This requires immediate testing of other animals to determine the degree of spread of the pathogen within the nucleus. Moreover, in order to maintain SPF status all infected animals, regardless of genetic merit or actual health (i.e. animals can carry a titer but remain healthy), must be culled from the nucleus. Consequently, under the traditional method, any genetic superiority possessed by these animals is forfeited.

In addition to the above costs, entire animal facilities need to be de-populated, sanitized and re-populated (but only after an appropriate waiting period). Even though the remaining SPF animals are usually limited to a few locations or age groups, the entire herd has to be re-stocked from this limited supply. This has two effects: (1) all or most of the animals are needed for re-stocking purposes. Consequently, little if any selection on genetic merit is possible. (2) Re-stocking a population from a subset of the population forces the population through a genetic selection “bottleneck” which decreases the number of sires and dams represented in the next generation thereby increasing the rate of inbreeding while simultaneously decreasing the genetic variation available for future selection. Such de-population/re-population occurrences can devastate the rate of genetic progress in a nucleus herd for an extended period of time and sharply reduce the rates of future genetic progress.

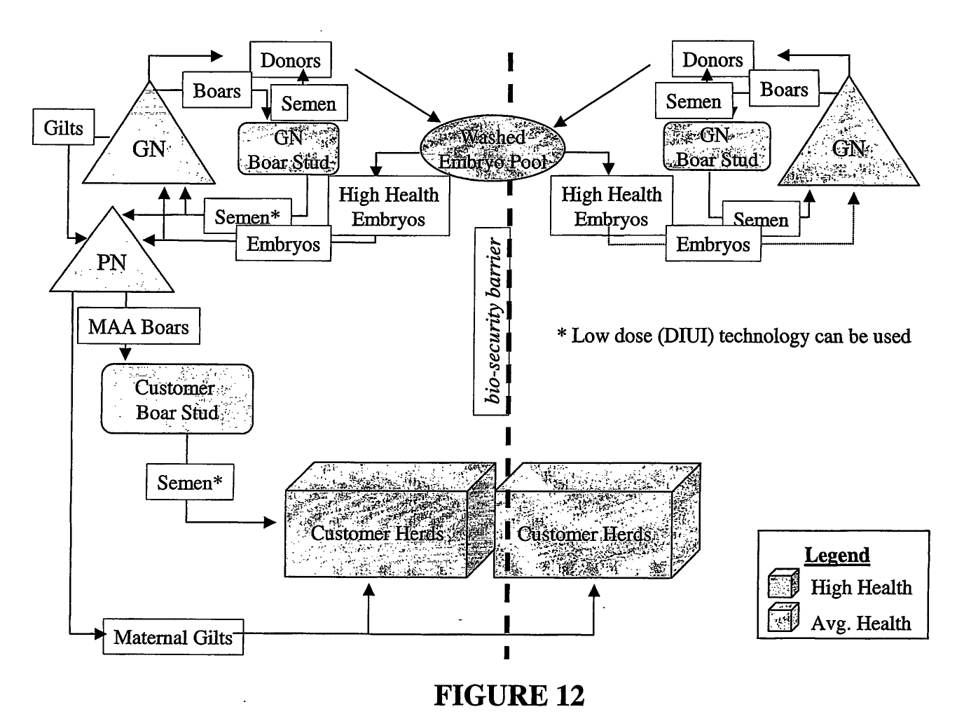

To overcome the deficiencies associated with currently available technologies, various embodiments of the instant invention provide methods that will allow breeding companies to deliver SPF germplasm via ET (combined with the “washing” of embryos prior to transfer) from populations that are carrying titers (i.e. populations that are infected but stable) for specific pathogens to SPF populations (see FIG. 12). For a detailed discussion of the process of “washing” embryos to remove any residual pathogenicity see, e.g. (National Hog Farmer, 2000, International. Pigletter, 2000

The instant invention provides strategic methods for using ET and MATE® (either independently or incorporated as an additional aspect of the “elite sire” methodology described supra) to produce SPF germplasm for sale to customers while concomitantly shielding the genetic nucleus from the usual decrease in rate of genetic progress caused by the protocols required to maintain SPF status (discussed supra). Various aspects of this embodiment of the invention obviate the need to cull genetically superior animals and also saves costs by eliminating the need to maintain strictly pathogen-free nucleus populations.

Another advantage of producing a pool of washed embryos, in accordance with the methods of the instant invention, is the ability to create a ready pool of SPF embryos, having high genetic quality, that can be safely transferred to other environments, including other genetic or production nucleus herds, without fear of transferring diseases. This is particularly helpful in the swine industry where nucleus herds in the same company have difficulty in safely moving germplasm within the company because the herd health status often varies with location. This phenomenon typically forces swine breeding companies to operate nucleus herds independently of each other for health reasons (one exception to this tendency is the transfer of semen originating from SPF boar studs). However, if embryos are routinely washed and handled according to the methods of the instant invention, they become a safely and easily transferable source of germplasm. Furthermore, breeding companies operating on a multi-national basis will also greatly benefit from being able to cheaply and safely move germplasm to foreign locations (e.g. production nuclei in several foreign countries).

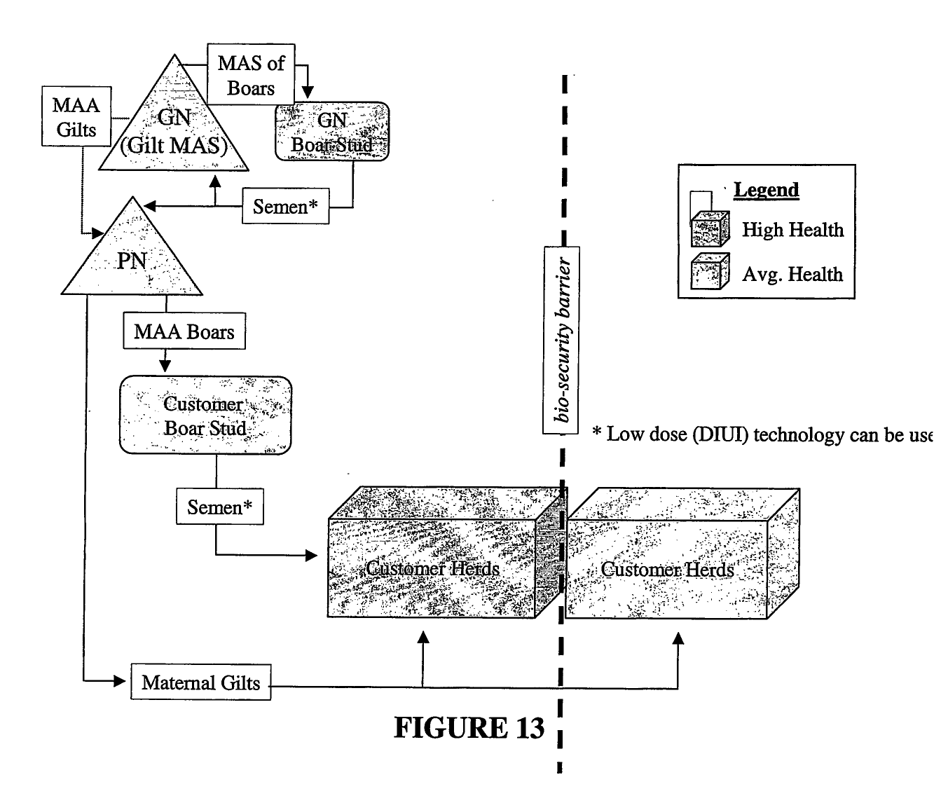

As described above, marker-assisted selection (MAS) and marker-assisted allocation (MAA) are methods (see FIG. 13) that utilize marker information that may be used in conjunction with various embodiments of the in instant invention. Further, since it is likely that animals in different genotype classes may have different breeding values or different management characteristics, it is likely that sorting the resulting offspring would be of interest. Therefore, marker information can be used to perform MAA early in the animal's life to allow sorting and optimizing the management of offspring. For example, a production nucleus manager could use the information to group females according to genotypic class. These females could then be mated strategically to provide offspring that have identical genotypes at several QTL (or ETL).

Similarly, a producer may want to use MAA to sort offspring for the purposes of optimizing management via diet and housing. Additionally, MAA could be used to target some groups of pigs for different production endpoints and/or particular marketing niches (e.g. the “table meat” vs. “lean yield” market). Furthermore, producers could further boost the genetic level of boars resulting by purchasing a greater number of embryos than are actually needed. In this manner, extra selection via MAS is possible early in life such that only the animals with the greatest genetic merit or a specifically desired genetic profile are kept, while the remainders are culled early in life to avoid unnecessary housing costs.

One of the difficulties associated with the use of MAS (and MAA) is the relatively small proportion of animals that coincidentally inherit the favorable alleles at all or most of the QTL (or ETL) that have been identified. This problem has been referred to as “limited selection space” (Soller, 2003). That is, if all the favorable QTL alleles are at low to moderate frequencies and QTL are segregating independently, the proportion of animals (P_{HO}) that are homozygous for the favorable allele at all (N) loci can be estimated according to Equation 1 as follows:

P_{HO}=(p₁)²(p₂)² . . . (p_{N})² (Equation 1)

where, p₁, p₂, through p_{N} are the frequencies of the favorable alleles at loci 1 through N. As can shown in Table 1, the proportion of animals homozygous for the favorable allele at all loci can be extremely low; particularly when the average frequency of favorable alleles is low to moderate or if the number of QTL is 5 or greater.

TABLE 1
Proportion of animals homozygous for favorable allele at all
loci given average frequencies (p_{Avg.})of favorable allele

| p_{Avg.} | 5 QTL | 10 QTL | 20 QTL |
|---|---|---|---|
| 0.3 | 5.90 × 10⁻⁶ | 3.49 × 10⁻¹¹ | 1.22 × 10⁻²¹ |
| 0.5 | 9.77 × 10⁻⁴ | 9.53 × 10⁻⁷ | 9.09 × 10⁻¹³ |
| 0.7 | 0.028 | 7.80 × 10⁻⁴ | 6.36 × 10⁻⁷ |

The effect of limited selection space is that in a typical nucleus herd population, the number of animals produced via regular selection is not large enough to find many (if any) animals that are homozygous at all loci for the favorable allele. For example, in a nucleus herd of 500 female animals each producing 20 offspring per year, 10,000 offspring can be produced. Despite this relatively large number of offspring, if the favorable allele is at a moderate frequency, say 0.5, only approximately 10 animals would be produced that are favorably homozygous at 5 loci and probably no animals would be produced when the goal is 10 or 20 QTL. The results are even more problematic when, as is often the case, favorable alleles tend to occur at lower frequencies.

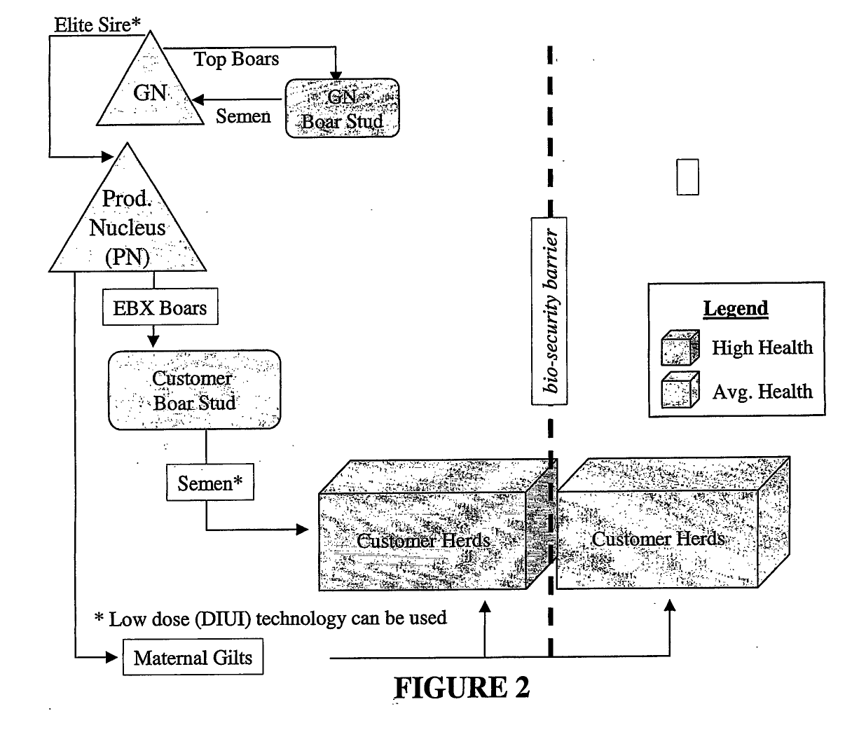

The instant invention, through its concerted use of the “elite sire” (FIG. 2) and MATE® strategies allows for the much more frequent production of those animals having the desired alleles at most or all selected QTL. As described above, one aspect of the “elite sire” method employs a target herd (e.g. a production nucleus) in addition to a genetic nucleus, not for the purpose of generating genetic progress, but rather to deliver germplasm to the customer with the highest genetic merit possible. Of particular relevance to the combination of the “elite sire” technology with the MATE® technology, is the ability to concentrate the use of a single or just a few sires, of a particular genetic profile, in the production nucleus (PN) greatly increase the probability that the offspring are of superior and more homogeneous in their genetic merit, as compared with the previous generation. That is, this method allows for the production of a large number of half-sibs from a single sire, thus greatly increasing the probability that offspring with the desired panel of QTL will be produced.

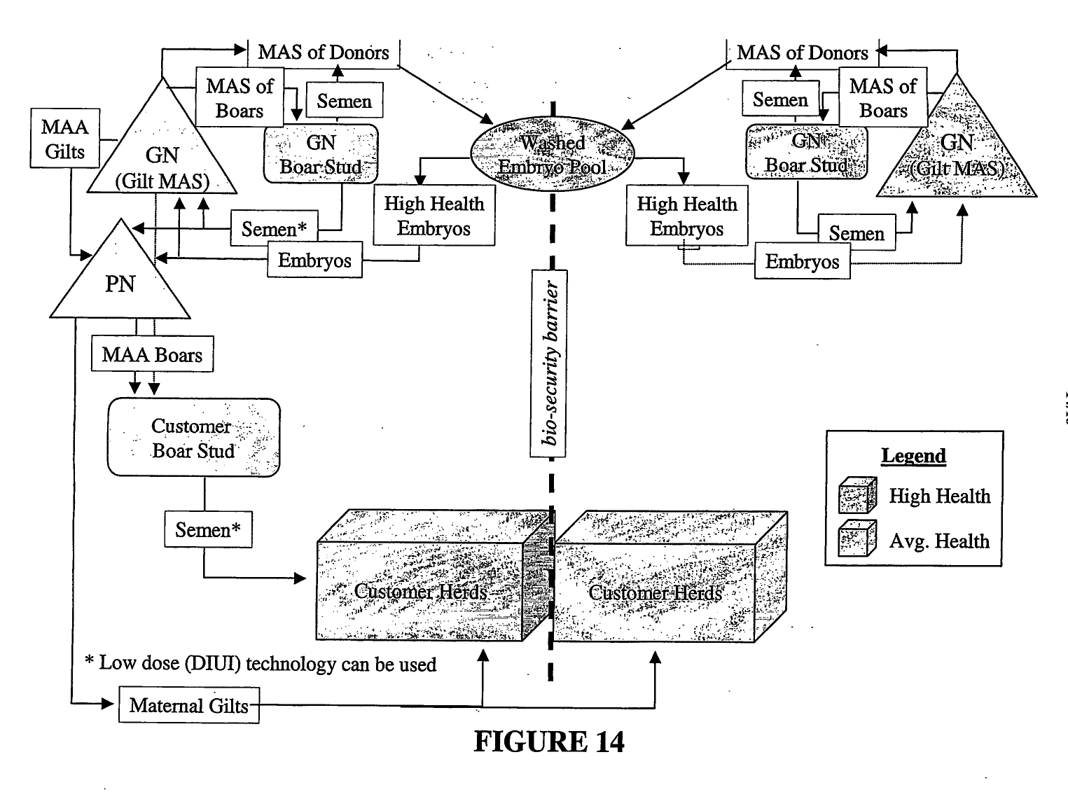

If ET and embryo washing is being employed to overcome the loss of genetic progress associated with SPF protocols, then several strategies become available to the nucleus herd manager. MATE®-Phase I, “strategic mating”, (FIG. 14) employs genetic markers during this process via MAS to more advantageously select the parents of ET offspring. This method provides for the strategic production of a larger group of animals having a particularly valuable genetic profile. The method also provides means for a more accurate ranking the resulting offspring in terms of their genetic merit. A particularly useful means for implementing this aspect of the invention is provided by the disclosure of U.S. provisional patent application Ser. No. 60/543,034, filed Feb. 9, 2004, which is incorporated herein by reference.

Using the previous example of a nucleus herd with 500 females each producing 20 offspring annually, we could predict that only 10, out of 10,000, resulting offspring would be favorably homozygous for 5 independent QTL with average frequency of favorable alleles was 0.5. Most likely, 5 offspring would be female and 5 male. Similar to this proportion of homozygotes, the proportion of animals (P_{HE}) that are heterozygous for the favorable allele at all (N) loci can be predicted as follows, using Equation 2:

P_{HE}=2(p₁)(1−p₁)2(p₂)(1−p₂) . . . 2(p_{N})(1−p_{N}) (Equation 2)

where, p₁, p₂ and p_{N} are the frequencies of the favorable alleles at loci 1 through N. As shown in Table 2, the proportion of animals heterozygous at all loci is still limited but higher than those homozygous at all loci as shown in Table 1.

TABLE 2
Proportion of animals heterozygous for favorable allele at all
loci given average frequencies (p_{Avg.})of favorable allele

| p_{Avg.} | 5 QTL | 10 QTL | 20 QTL |
|---|---|---|---|
| 0.3 | 0.013 | 1.71 × 10⁻⁴ | 2.90 × 10⁻⁸ |
| 0.5 | 0.031 | 9.76 × 10⁻⁴ | 9.53 × 10⁻⁸ |
| 0.7 | 0.013 | 1.71 × 10⁻⁴ | 2.90 × 10⁻⁸ |

Therefore, in our example, if 5 male and 5 female offspring could be found that are homozygous for the 5 loci, then 310 animals (or 155 females) should be available that are heterozygous. If the homozygous males were mated to the heterozygous females, then 50% of the resulting offspring would now be homozygous at all 5 QTL. This is considerably higher than matings planned without the knowledge of the QTL genotypes, which would be similar to the proportions given in Table 1. However, selection in a genetic nucleus is not likely to based solely on the genotypes of 5 QTL, therefore, it would be expected that other factors such as other QTL, phenotypic, and pedigree information would also be included in the selection decision. In addition, the inclusion of more sires per generation may be considered to limit the increase in inbreeding rate. Nevertheless, frequencies of favorable alleles at key QTL could be increased more quickly via the strategic use of ET in the GN in accordance with the instant invention.

One remarkable advantage of this kind of strategic mating is in the context of transferring germplasm from a genetic nucleus to a production nucleus. Various aspects of the instant invention provide the opportunity to create more genetic homogeneity in higher volumes than is possible using unplanned matings. For instance, by employing multiple ovulation ET (MOET) to mate selected individuals, up to 10 litters can be produced each year from a single female. Assuming that the litter sizes for such matings are similar to AI litters and other that other surrogate females are used as recipients, 90-100 offspring could be produced annually from each donor female. In this example, only 6-7 ET sessions per donor would be required annually for a 155 selected females to produce the 10,000 offspring that would normally require 500 breeding females via regular AI. The result would be 10,000 offspring that would be homozygous at half of the loci included in the selection process. The proportion of these animals that would be homozygous at all 5 loci would be equal to 0.5^{N} where N is the number of loci included in the mating scheme. Therefore, the number of offspring that are homozygous for favorable alleles is now equal to the proportions given in Table 3 instead of those in Table 1.

TABLE 3
Proportion of animals homozygous for favorable alleles
at all loci assuming one parent is homozygous and
the other is heterozygous at all loci

| 5 QTL | 100 QTL | 20 QTL |
|---|---|---|
| 0.031 | 9.76 × 10⁻⁴ | 9.53 × 10⁻⁸ |

Under the conditions given, the number of favorably homozygous offspring is now 310 instead of 10, an approximately 31-fold increase. When the original favorable allele frequencies 0.3 instead of 0.5, this mating strategy results in 5200-fold increase in the proportion of animals favorably homozygous at all loci.

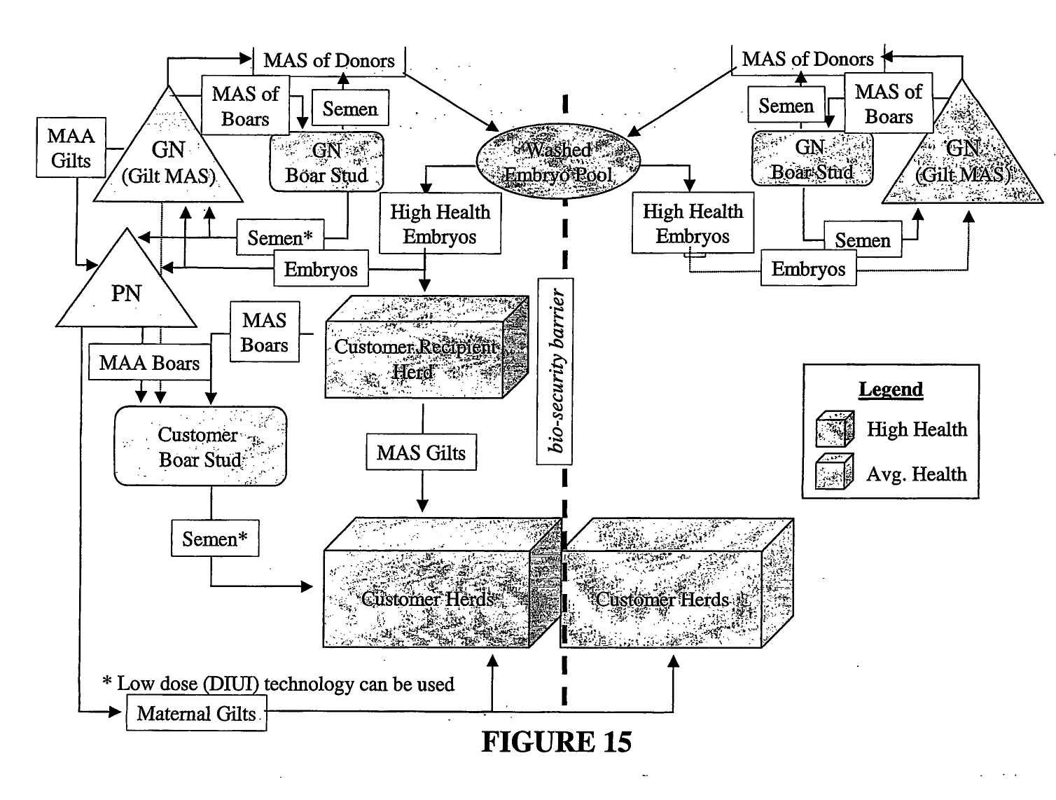

Another embodiment of the present invention is termed the MATE®-Phase II or “Direct Delivery” solution. Various aspects of this embodiment, provide advantages in addition to transferring the embryos to a production nucleus location, these advantages include transferring embryos directly from a GN herd to a customer recipient herd location via the use of a washed embryo pool (FIG. 15). By eliminating the production nucleus generation, elite germplasm can be brought to the customer more quickly. This “Direct Delivery” would be even more effective if IVF was used to increase the number of embryos harvested from each elite donor female. Utilizing this embodiment of the invention, the customer(s) could improve their bio-security further by purchasing SPF embryos rather than boars. This option eliminates the risk that a breeding company's boar facility may have become infected without the infection being detected for a period of time. Aspects of this embodiment of the invention could be used when customers decide to upgrade their herds by integrating a new source of maternal genetics but does not want to take the risk of introducing any new pathogens. The SPF embryos would be free of all pathogens and would therefore assume the health status of the customer's recipient herd. If this herd has a high-health status, there is no risk of introducing new pathogens and, therefore, there is no need to perform the costly de-population and re-population protocol usually employed when switching maternal-line genetics. Further, if the customer so desired, the breeding company could “sex” the embryos prior to delivery to guarantee that only males are delivered.

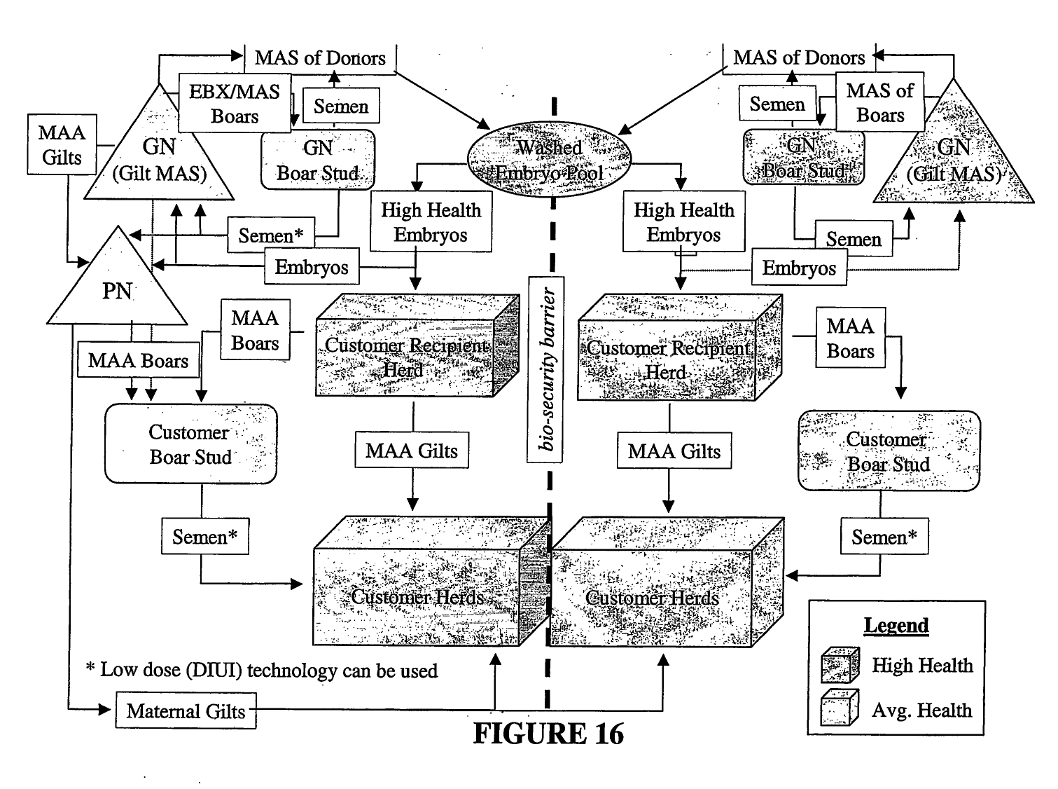

A further embodiment of the present invention is termed the MATE®-Phase III “Immuno-Competent” solution. In most cases in the swine industry, producers cannot justify maintaining an SPF environment. More common, is the scenario in which a producer herd is infected with one or more pathogens but the herd has developed a stable health status, in which most animals in the herd have been exposed to the pathogen (e.g. a particular strain of PRRSV) and have developed an relatively reasonable tolerance such that economic losses are minimized. In MATE®-Phase III, SPF embryos could be transferred into immuno-competent recipients FIG. 16). By doing this, the developing fetus passively acquires immunity from the recipient mother. This strategy is superior to one in which SPF animals (with no immuno-competence) are introduced into a pathogen-stable environment. In the latter scenario, new animals would not be prepared to tolerate the pathogen resulting in considerable economic losses. As with MATE®-Phase II, de-population of the producer's herd would not be required when introducing a new source of breeding stock. For producer's that would prefer to switch to a superior source of maternal-line germplasm, but hesitate due to the de-population costs, this aspect oft the instant invention provides an ideal solution.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### Example 1

### Fixing of Desired FUT1 Allele in a Herd Population

The first example illustrates the power of the instant invention for changing gene frequency in a population can be shown using the FUT1 gene. The FUT1 (alpha (1,2) fucosyltransferase 1) gene controls resistance and susceptibility to E. coli F18 adhesion to the mucosa of the small intestine. There are two forms (alleles) of the gene. The dominate allele (symbolized by “S”) codes for fimbrial receptors for the E. coli F18 fimbria, resulting in proliferation of that particular strain of E. coli in the small intestine of the pig and creating disease characterized by severe diarrhea and edema (Vogeli et al., 1999). The recessive allele (symbolized by “r”) codes for the absence of such fimbrial receptors, thereby conferring resistance to the E. coli F18 as well as to clinical signs of disease caused by the bacteria. In terms previously described, the “desired trait” is improved health or absence of disease and “improved germplasm” is the presence and/or frequency of animals in the population with the homozygous recessive genotype (“rr”).

Assuming the recessive allele exists at an intermediate frequency in the population (e.g. 0.50), then the expected frequency of animals with the improved germplasm occurring at random in the population is 25%. Selecting an elite sire with the improved germplasm and mating the elite sire to a target herd with the same intermediate frequency would produce a population of progeny having the improved germplasm in 50% of the animals, a doubling of the frequency in just one generation. Furthermore, if female progeny of the elite sire having the “rr” genotype were selected as replacement gilts for the target herd, and a second elite sire having the “rr” genotype was selected to produce the next generation, then the “rr” genotype could be fixed in the target herd in the second generation. In the case of terminal boar production, this would result in a new commercial boar product with the improved germplasm in 100% of the animals in just two generations without needing to select for the gene in the GN herd.

Using this same example and assuming the recessive allele exists at a very low frequency (e.g. 0.05) in the initial population, then the expected frequency of animals with the improved germplasm occurring at random in the population is approximately 0.25%. Using the same breeding strategy described above, 5% of the animals in the first generation of elite sire progeny would have the “rr” genotype (a twenty-fold increase in the frequency of animals with the improved germplasm). In the second generation, 52.5% of the elite sire progeny would have the improved germplasm and in the third generation, the “rr” genotype could be fixed. This is example of change in gene frequency clearly illustrates the power of the instant invention, given the necessary circumstances.

### Example 2

### Improvement of Body Weight at 196 Days of Age

The second example illustrates the use of the instant invention for improving body weight at 196 days of age (BWT196), an efficient growth trait. This is a quantitative trait that reflects an animal's potential for growth rate (increased body weight per unit of time). It is a phenotypic trait that can be measured on each animal in the population and the resulting data approximates a normal, bell-shaped curve. Trait expression (i.e. phenotypic value) is the result of a combination of both genetic and environmental effects. Genetic control of the trait is not completely known, but it is assumed that many genes with effects of varying sizes interact both additively and non-additively to create the cumulative genetic effect.

Two methods previously described that are used to estimate an animal's genetic potential to pass on improved phenotype to its progeny are BLUP and MA-BLUP. These methods use phenotypic data (e.g. BWT196) and pedigree information along with genotypic data (MA-BLUP only) to compute estimated breeding values (EBV). Like phenotypes, EBV's are distributed in a normal, bell-shaped curve. For purposes of this example, it will be assumed that EBV's are from a distribution with mean of zero and standard deviation of 5 lb. In terms previously described, the “desired trait” in this case could be defined as an EBV for BWT196 that is at least 10 lb (two standard deviations) greater than the current population mean, and the “improved germplasm” would be the underlying genetic effects (known or unknown) that contribute to improved BWT196.

It would be expected (based on normal distribution theory) that in a population with a mean of zero and a standard deviation of 5 lb., approximately 2.3% of the animals would have the desired trait (EBV of 10 lb or greater). It would also be reasonable to identify a single elite sire each generation from the GN with an EBV that is at least 15 lb greater than the average EBV in the population. Selecting an elite sire from the GN with an EBV of 15 lb and mating the elite sire to a target herd with the same mean EBV as the GN would produce a population of progeny having a mean EBV of 7.5 lb and having approximately 30% of animals with the desired trait, a thirteen-fold increase in the first generation. Furthermore, if female progeny of the elite sire having the desired trait were selected as replacement gilts for the target herd, and a second elite sire having an EBV of 15 lb was selected to produce the next generation, then the mean of the target herd in the second generation would be EBV of 12.5 lb and having approximately 70% of animals with the desired trait.

Unlike the first example, selection in the second example is on a desired trait rather than the actual improved germplasm. It is assumed that the EBV's do indeed reflect actual genetic differences and that improved germplasm will be the result. As MA-BLUP replaces BLUP and more information is known about individual genes and their effects on traits of economic interest, EBV's for quantitative traits will become more accurate and there will be little difference in selecting for a “desired trait” or selecting for an “improved germplasm”. But for purposes of this disclosure the two are distinctly different and equally important illustrations of how the instant invention may be used to create improved products.

### Example 3

### Modulation of MC4R to Improve Feeding Behavior and Body Weight

Similar to Example 1, selection of animals can be enhanced by the inclusion, during the execution of the described methods, of data pertaining to candidate genes that have been verified to have a proven effect in the population being selected. Such a gene is the porcine melanocortin-4 receptor (MC4R). Mutations in the MC4R gene have been implicated in the regulation of feeding behavior and body weight in humans and mice. Moreover, missense variants in this gene have been shown to have significant associations with backfat thickness and growth rate in several lines of pigs (see Kim et al., 2000). In the Kim et al. studies, the “11” genotype was found to result in 9% less backfat than the “22” genotype, whereas pigs with the “22” genotype grew significantly faster (˜37 g/day faster) than the pigs with the “11” genotype. These results appear to be a function of appetite because the “22” genotype consumed an average of 0.17 kg/day more feed than the “11” genotype. More recently, another study (Hernandez-Sanchez et al., 2003) supported the earlier findings in several lines with the exception that the allele definitions appear to have been reversed.

The methods of the instant invention provide useful means enabling the rapidly change the frequencies of the MC4R gene in the target population; thereby providing an immediate boost in growth rate or decrease in backfat. A particularly useful application of the instant invention is the allocation of animals having different genotypes to different customers. Whereas the genetic nucleus population would tend to follow a long-term selection strategy that would favor one of the alleles (e.g. selection of the “2” allele to increase growth rate), individual customers (e.g. European customers facing higher feed prices) may prefer to select for the “1” allele to reduce backfat and improve lean tissue feed conversion efficiency. Alternatively, feed prices could skyrocket in response to a short-term weather change that would cause customers to temporarily prefer animals that carry the “1” allele. The invention allows the GN selection to proceed with selection consistent with long-term goals, but quickly and temporarily increase the frequency of the “1” allele in the target population.

### Example 4

### Modulation of IGF2 to Modulate Muscle Mass and Backfat

Another example demonstrates the use of imprinted genes or QTL, such as the QTL mapped to the IGF2 locus on pig chromosome 2p, to modulate herd characteristics. The IGF2 QTL was found to be associated with significantly increased muscle mass and simultaneously decreased backfat (Nezer et al., 1999 and Jeon et al., 1999). This QTL has been referred to as the “BETTERgen muscle+” gene by Seghers Genetics and in a patent titled “Selecting Animals for Parentally Imprinted Traits” (PCT: WO 00/36143), which is herein incorporated by reference in its entirety. Of particular interest was the finding that this QTL appears to be paternally imprinted, i.e. the effect of the favorable QTL allele was only expressed if inherited from the sire. This is especially useful to swine breeding companies because the germplasm is most cost-effectively transferred to the customer via semen or live boars. With paternally imprinted genes, the maternal genotype becomes unimportant since is not expressed in the offspring. In this case, since lower backfat has been associated with poorer fertility. Thus, it would likely be particularly advantageous to limit the use this QTL to terminal boar lines and select for the opposite allele in the maternal line or perhaps avoid selection at this locus altogether using the instantly described methods.

### Example 5

### The CHOICE ADVANTAGE SYSTEM^{SM}

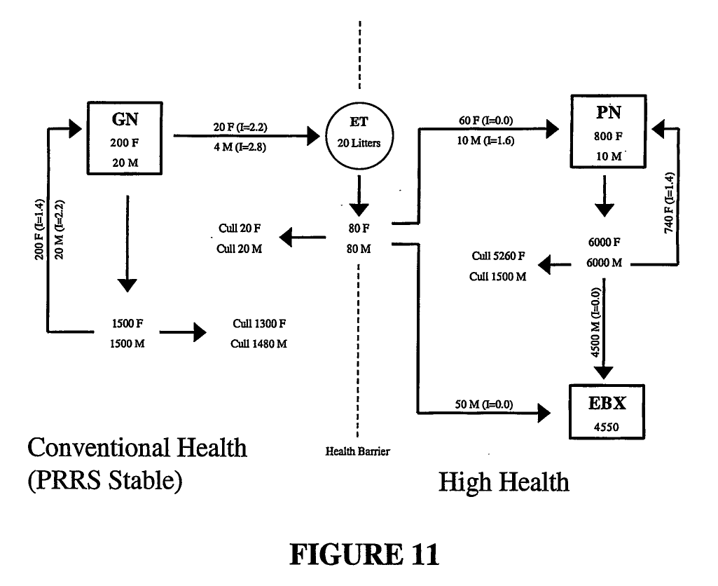

FIG. 11 depicts a method for genetic improvement of terminal sires, known as the CHOICE ADVANTAGE SYSTEM^{SM}. This system leverages the use of a few (e.g. 10) elite sires produced in the GN for the production of a large number of terminal (EBX) boars in the PN. Furthermore, the elite sires from the GN are produced via ET and/or IVF in order to move the elite embryos across an existing health barrier. In addition, the embryos are produced from an intensely selected subset of the existing males and females in the GN herd. These parents are allocated for matings using marker assisted allocation (MAA), a combination of phenotypic and genotypic information. One specific aspect of this the process is marker-assisted embryo transfer (MATE). This process may also be combined with DIUI to allow the use of a yet smaller number (1 to 9) of elite sires at the PN level.

This process may also be used to produce large numbers of progeny from elite dams through super ovulation and in vitro fertilization or other similar reproductive technologies. One step in this process where it would be particularly beneficial to produce many progeny from a single elite dam would be in the production of embryos from the GN herd. This would allow many litters to be produced from the same sire and dam, thereby greatly increasing the probability of producing a desirable elite sire or sires.

Another step in the process where the use of elite dams would be beneficial is in the composition of the PN herd itself. If only a fraction of the 800 sows in the example were needed as dams of the EBX boars, then selection intensity of those dams would be greatly increased, resulting in more EBX boars with the desirable traits or attributes. Although this would not necessarily reduce the number of females needed to raise the EBX boars, it would reduce the number of females needed as serve as genetic mothers of the EBX boars.

The methods described herein are particularly well suited to allow swine producers to more fully exploit the beneficial attributes of imprinted genes or QTL such as IGF2. Moreover, there may be advantages to not selecting an entire line for the lean growth allele, but rather raising or fixing the frequency of the favored allele quickly in a target herd population while maintaining the favorable allele at moderate frequencies in a GN herd. This may be especially advantageous if the imprinted allele has beneficial effects in the terminal animal (i.e. slaughter pig) but also has some negative impact, or is linked to genes with negative impact, on the breeding population (e.g. reduced fertility). If this were the case, the breeding company would prefer to only raise the frequency of the lean growth allele in the GN herd to the level required to produce sufficient terminal sires for intensive use in the target herd population, where the gene could be fixed or raised to high frequency. In this way, the breeding efficiency of the GN herd would not compromised. Alternatively, if maternal traits were severely affected by the imprinted gene, a breeding company might prefer to only fix the imprinted gene amongst the elite sires to be used in the target herd.

Finally, while MAS applied using information on identified loci or QTL allows faster genetic response in the early generations, this is often done at the expense of polygenic response, which is not entirely recoverable in later generations (Gibson, 1994; Pong-Wong and Woolliams, 1998). This is so because exerting the selection pressure required to select the particular marker sought causes certain portions of the genomes of the animals possessing those markers to be fixed in the herd population. As a result, genetic variance is lost not only at the particular markers selected for, but also at those portions of the genome associated with the selected markers.

Optimal control theory has been applied to mitigate this loss of genetic variation problem by optimizing long-term selection response over several generations via controlling the selection weights placed on the QTL (Dekkers and van Arendonk, 1998; Dekkers and Chakraborty, 2001; Chakraborty et al., 2002). Unfortunately, application of this theory results in relatively slow increases in the frequency of the favorable QTL allele. Moreover, the greater amount of time needed to increase the selected QTL alle's prevalence to the desired frequency is often problematic for breeding companies because short-term market share can be heavily influence by the availability of animals with favorable QTL alleles. Consequently, the instant invention advantageously allows for relatively stringent QTL selection in the target herd with concomitant use of optimal control theory to maximize long-term response in the GN. Accordingly, if marketing reasons exist for increasing the frequency of favorable QTL alleles even faster than in standard QTL selection, intense short term selection pressure can be applied on certain genes and QTL in the target herd while maintaining an optimal long-term selection pressure on the same genes and QTL in the GN herd. Thus one great advantage provided by the instant invention is that its methods allow intense short term selection pressure to be applied on certain genes and QTL in the target herd while simultaneously optimizing selection pressure on these genes and QTL in the GN herd.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention as defined by the appended claims.

### REFERENCES

The following references, to the extent that they provide exemplary procedural or other details supplementary to those set forth herein, are specifically incorporated herein by reference.
Animal Breeding: Use of New Technologies, Kinghorn, B., van der Werf, J., and Ryan, M., Eds., published by The Post Graduate Foundation in Veterinarian Science of the University of Sydney, Sydney, Australia, 1999.
Embryo Transfer Preserves Genetics, Sidesteps PRRS, National Hog Farmer, Nov. 15, 2000.
International embryo transfer: It can be done!, International Pigletter Vol. 20 No. 10, December 2000.
Chakraborty, R., Moreau, L., Dekkers, J. C. 2002. A method to optimize selection on multiple identified quantitative trait loci. Genet. Sel. Evol. 34(2):145-70.
Dekkers, J. C. M. and van Arendonk, J. A. M. 1998. Optimizing selection for quantitative traits with information on an identified locus in outbred populations. Genet. Res. 71:257-275.
Dekkers, J. C. M. and Chakraborty, R. 2001, Potential gain from optimizing multigenerational selection on an identified quantitative trait locus. J. Anim. Sci. 79:2975-2990.
Garcia, E. T. et al., U.S. Pat. App. Pub. No.: U.S. 2002/0072650 A1, published Jun. 13, 2002.
Gibson, J. P. 1994. Short-term gain at the expense of long-term response with selection of identified loci. Proc. 5^{th} World Cong. Genet. Applied Livest. Prod., Guelph, Canada. 21:201-204.
Falconer, D. S. and Mackay, Introduction to Quantitative Genetics, T.F.C., Eds., Longman Group Limited, Longman House, Burnt Mill, Harlow Essex 2JE, England. 4^{th} Edition, 1986.
Fernando, R. L. and Grossman, M. 1989. Marker assisted selection using best linear unbiased prediction. Genet. Sel. Evol. 21: 467-477.
Hazeleger, W., “Assembly and method for penetrating the uterus of an animal during a non-surgical procedure”, WIPO international Pub. No. WO 99/27868, Jun. 10, 1999.
Henderson, C. R. 1959. The estimation of genetic and environmental trends from records subject to culling. Biometrics 15:192-218.
Henderson, C. R. 1963. Selection index and expected genetic advance. Hanson W. D. and Robinson, H. F., Eds., Statistical Genetics and Plant Breeding, pp. 141-163. Publ. 92, National Academy of Sciences, National Research Council, Washington, D.C.
Henderson, C. R. 1976. A simple method for computing the inverse of a numberator relationship matrix used in prediction of breeding values. Biometrics 32:69-83.
Henderson, C. R. 1984. Applications of Linear Models in Animal Breeding. Published by the University of Guelph, Guelph, Ontario, Canada.
Hernandez-Sanchez, J., Visscher, P., Plastow, G. and Haley, C. 2003. Candidate Gene Analysis for Quantitative Traits Using the Transmission Disequilibrium Test: The Example of the Melanocortin 4-Receptor in Pigs. Genetics. 164:637-644.
Hoeschele, I. 1993. Elimination of quantitative trait loci equations in animal models incorporating genetic marker data. J. Dairy Sci. 76:1693-1713.
Jeon, J. T., Carlborg, O., Tornsten, A., Giuffra, E., Amarger, V., Chardon, P., Andersson-Elklund, L., Andersson, K., Hansson, I., Lundstrom, K. and Andersson, L. 1999. A paternally expressed QTL affecting skeletal and cardiac muscle mass in pigs maps to the IGF2 locus. Nature Genetics. 21:157-158.
Kim, K. S., Larsen, N., Short, T., Plastow, G. and Rothschild, M. F. 2000. A missense variant of the porcine melanocortin-4 receptor (MC4R) gene is associated with fatness, growth, and feed intake traits. Mammalian Genome. 11:131-135.
Martinez, E. A., Vazquez, J. M., Roca, J., Lucas, X., Gil, M. A., Parrilla, I., Vazquez, J. L., and Day, B. N., “Minimum number of spermatozoa required for normal fertility after deep intrauterine insemination in non-sedated sows,” Reproduction 123:163-170, 2002).
Martinez, E. A., Vazquez, J. M., Roca, J., Lucas, X., Gil, M. A., Parrilla, I., and Vazquez, J. L., “Deep intrauterine insemination and embryo transfer in pigs,” Reproduction Supplement 58:301-311, 2001.
Melecot, G., Les mathématiques de l'Hérédité, Masson and Cie, Paris, 1948.
Meuwissen, T. H. E. 1996. The use of marker haplotypes in animal breeding schemes. Genet. Sel. Evol. 28: 161-176.
Nezer, C., Moreau, L., Browers, B., Coppieters, W., Detilleux, J., Hanset, R., Karim, L., Kvasz, A., Leroy, P. and Georges, M., 1999. An imprinted QTL with major effect on muscle mass and fat deposition maps to the IGF2 locus in pigs. Nature Genetics. 21:155-156.
Pong-Wong, R. and Woolinams, J. A. 1998. Response to mass selection when an identified major gene is segregating. Genet. Sel. Evol. 30:313-337.
Quaas, R. L., Anderson, R. D., Gilmour, A. R., 1984. BLUP school handbook; Use of mixed models for prediction and estimation of (co)variance components. Animal Breeding and Genetics Unit, University of New England, N.S.W. 2351, Australia.
Quaas, R. L. 1988. Additive genetic model with groups and relationships. J. Dairy Sci. 71:1338-1345.
Rath, D., “Low dose insemination in the sow—a review”, Reprod. Domest Anim. 37:201-5, 2002.
Ruane, J. and Colleau, J. J. 1995. Marker assisted selection for genetic improvement of animal populations when a single QTL is marked. Genet. Res., Camb. 66, pp. 71-83. Cambridge University Press.
Ruane, J. and Colleau, J. J. 1996. Marker-assisted selection for a sex-limited character in a nucleus breeding population. J. Dairy Sci. 79:1666-1678
See, M. T., “Intrauterine (transcervical) and fixed-time artificial insemination in swine: a review,” SWINE NEWS North Carolina Cooperative Extension Service, Vol. 25, no. 2, March 2002.
Singh, E. Embryo Transfer: An assessment of the risks of disease transmission. In: Managing Global Genetic Resources. The Committee on Managing Global Genetic Resources: Agricultural Imperative. National Academy Press, Washington, D.C., 1993, pp 171-231.
Soller, M. 2003. Proceedings of the John M. Airy Genetic Visions Symposium, Johnston, Iowa.
Totir, L. R. 2002. Genetic evaluation with finite locus models. PhD Dissertation. Iowa State University, Ames, Iowa.
Van Arendonk, J. A. M., Smith, C. and Kennedy, B. W. 1989. Method to estimate genotype probabilities at individual loci in farm livestock. Theor. Appl. Genet. 78: 735-740.
Van Vleck, L. D. 1983. Notes on the Theory and Application of Selection Principles for the Genetic Improvement of Animals. Department of Animal Science, Cornell University. Ithaca, N.Y.
Van Vleck, L. D., Pollak, E. J., and Oltenacu, E. A. B., Genetics for the Animal Sciences, W.H. Freeman and Company, New York, 1987
Verrier, E. 2001. Marker assisted selection for the improvement of two antagonistic traits under mixed inheritance. Genet. Sel. Evol. 33: 17-38
Vogeli, P., Meijerink, E., Stranzinger, G. and Bertschinger, H. U. 1999. Porcine Escherichia coli diarrhoea and oedema disease: pathogenesis and identification of genetic resistant breeding stock. Arch. Tierz. 42: 28-35.
Wang, T., van der Beek, S., Fernando, R. L. and Grossman, M. 1991. Covariance between relatives for a marked quantitative trait locus. Theor. Appl. Genet. 27:251-274.
Wang, T., Fernando, R. L. and Grossman, M. 1998. Genetic evaluation by BLUP using marker and trait information in a multibreed population. Genetics 148:507-515.
Wang, T., Fernando, R. L., Stricker, C. and Elston, R. C. 1996. An approximation to the likelihood for a pedigree with loops. Theor. Appl. Genet. 93: 1299-1309.
Watson, P. F., Behan, J., Decuardro-Hansen, G., and Cassou, B., VI^{th} International Conference on Pig Reproduction, University of Missouri-Columbia, p. 135, 2001.
Weller, J. I. 2001. Quantitative Trait Loci Analysis in Animals. CABI Publishing, 10 E 40^{th} St., Suite 3203, New York, N.Y. 10016 USA

1. A Method for producing terminal swine parent animals having improved germplasm, the method comprising:
a. providing at least one genetic nucleus herd and/or a target herd for which improvement is desired
b. selecting a trait or traits, for which improvement is desired;
c. providing semen aliquots from an elite sire selected from the genetic nucleus (GN) herd wherein the elite sire has a desired germplasm that is determinative for improving one or more selected trait(s) in the target herd;
d. using the semen aliquots to impregnate a correlative number of breeding females in a target herd; wherein the semen from the elite sire is used to breed substantially all of the females in the target herd;
e. producing half-sib offspring having improved germplasm when compared with the breeding females in the target herd; and
f. providing at least one of the half-sib offspring as the terminal swine parent in a SP (swine production herd), or as a replacement animal for the GN herd, or as a replacement animal for the target herd, whereby the genetics are improved in the target herd and/or SP.

2. The method of claim 1 wherein the herd(s) for which improvement is desired is selected from one or more of the group consisting of: a genetic nucleus herd, a production nucleus herd, crossbred boar multiplier herd, an external genetic nucleus herd, and a crossbred gilt multiplier herd.

3. The method of claim 2 wherein the target herd is a production nucleus herd.

4. The method of claim 2 wherein the target herd is a crossbred boar multiplier herd.

5. The method of claim 2 wherein the target herd is a crossbred gilt multiplier herd.

6. The method of claim 2 wherein the target herd is an external genetic nucleus herd.

7. The method of claim 1 wherein the selected elite sire is selected for as having germplasm favorable for providing offspring having at least one of the following: one or more desired qualitative or economic trait locus/loci; one or more desired quantitative trait locus/loci a desired estimated breeding value (EBV); a desired genotype or phenotype; one or more desired health trait(s), one or more desired meat quality trait(s), one or more desired reproduction trait(s); or one or more desired efficient growth trait(s).

8. The method of claim 1 comprising identifying female half-sib offspring having preferred germplasm and retaining these female half-sib offspring as breeding females in the target herd.

9. The method of claim 1 comprising:
a. providing semen aliquots from an elite sire selected from a GN (genetic nucleus herd), the elite sire having a germplasm determinate for a SETBV (specific economic trait breeding value);
b. using the semen aliquots to impregnate a correlative number of breeding females in a target herd, wherein the breeding females have a lower SETBV than the elite sire and wherein the semen aliquots are used according to a breeding plan designed to be suitable to produce an average of at least 160 half-sib offspring per week, from the elite sire, for a period of from one week to about one year
c. producing half-sib offspring having a higher SETBV than the breeding females in the target herd; and
d. providing at least one of the half-sib offspring for use as a terminal swine parent in a SP (swine production herd) or as a replacement animal in the target herd; whereby the average SETBV is increased in the target herd and/or the SP herd.

10. The method of any of claims 1-8 wherein semen from the elite sire is used in a breeding plan suitable for producing an average of at least 160 half-sib offspring per week from the elite sire, for a plurality of successive weeks.

11. The method of claim 1 wherein semen from the elite sire is used in a breeding plan is suitable for producing an average of at least 320 half-sib offspring per week from the elite sire, for a plurality of successive weeks.

12. The method of claim 1 wherein semen from the elite sire is used in a breeding plan is suitable for producing an average of at least 640 half-sib offspring per week from the elite sire, for a plurality of successive weeks.

13. The method of claim 1 wherein semen from the elite sire is used in a breeding plan is suitable for producing an average of at least 1280 half-sib offspring per week from the elite sire, for a plurality of successive weeks.

14. The method of any of claims 1-8 wherein the germplasm is improved in the target herd independently of any improvement in the GN herd.

15. The method of any of claims 1-8 wherein the germplasm is improved in the SP herd independently of improvement in the GN herd.

16. The method of any of claims 1-8 wherein the germplasm is improved in the target herd and/or SP concurrently with improvement in the GN herd.

17. The method of any of claims 1-8 wherein the germplasm is improved more rapidly in the target herd and/or SP herd than in the GN herd.

18. The method of claim 1 wherein the breeding females are impregnated using deep intrauterine insemination (DIUI).

19. The method of 18 wherein the DIUI is conducted using 1.5 billion or fewer spermatozoa.

20. The method of claim 18 wherein the DIUI is conducted using between 75 million and 1.5 billion spermatozoa.

21. The method of claim 18 wherein the DIUI is conducted using between 30 million and 1.5 billion spermatozoa.

22. The method of claim 18 wherein the DIUI is conducted using between 10 million and 1.5 billion spermatozoa.

23. The method of claim 18 wherein the DIUI is conducted by delivering the spermatozoa to the anterior portion of the uterine horn.

24. The method of claim 1 wherein the breeding females are inseminated using 75 million, or fewer spermatozoa.

25. The method of claim 1 wherein the breeding females are impregnated using 10 to 75 million spermatozoa.

26. The method of claim 24 wherein the insemination is conducted by delivering the spermatozoa at or near the utero-tubal junction.

27. The method of claim 1 wherein a single boar is used to impregnate all or substantially all of the breeding females in the target herd.

28. The method of claim 1 wherein the GN herd is Rendement Napole-negative and halothane-negative.

29. The method of claim 1 wherein the GN herd is maintained under selection pressure at a population size effective for maximizing rate of genetic improvement according to a breeding plan while simultaneously maintaining an acceptable rate of inbreeding.

30. The method of claim 1 comprising selecting, for use as terminal sires, male half-sib offspring having the desired germplasm.

31. The method of claim 1 comprising making rapid genetic improvement in commercially valuable traits in the target herd and in the terminal sires for the SP herd through use of a breeding plan designed to provide an improvement selected from the group consisting of (1) increasing the frequency of one or more desirable alleles for commercially valuable traits that contribute to higher estimated breeding values, (2) optimizing the frequencies of certain alleles or genotypes known to be associated with quantitative trait loci (QTL) for commercially valuable traits; and (3) optimizing the frequencies of certain alleles or genotypes for several qualitative economic trait loci (ETL) linked to commercially valuable traits, and combinations of any two or three thereof.

32. The method of claim 1 wherein the selected trait(s) include one or more of the following:
efficient growth traits; meat quality traits; reproduction traits; and health traits.

33. The method of claim 1 comprising selecting female half-sib offspring to be replacement dams in the target herd and further comprising repeating steps a. through d. to achieve a desired level and/or frequency of the selected trait or traits in the target herd.

34. The method of claim 1 wherein an improvement of the selected trait or traits is/are achieved in the target herd without introducing any improvement of these trait(s) into the GN herd.

35. The method of claim 1 wherein the selected trait(s) are improved to a more favorable degree in the target herd than in the GN herd.

36. The method of claim 1 wherein the frequency of one or more alleles is fixed in the target herd without being fixed in the GN herd.

37. The method of claim 1 wherein, by using a target herd separate and distinct from the GN herd, the desired trait(s) are improved separately in the target herd as compared with the GN herd through use of one or more of the following procedures: (1) using a GN herd containing the minimum number of females necessary to maintain high annual genetic gain and low annual change in inbreeding level or (2) using a target herd containing the minimum number of females to meet plan-specified market demands for target herd offspring.

38. The method of claim 1 wherein the trait(s) selected for improvement comprises one or more traits selected from the group consisting of: efficient growth traits; meat quality traits; reproduction traits; and health traits.

39. The method of claim 1 wherein at least one selected trait is an efficient growth trait selected from the group consisting of: average daily gain, average daily feed intake, feed efficiency, back fat thickness, loin muscle area, and lean percentage.

40. The method of claim 1 wherein at least one selected trait is a meat quality trait selected from the group consisting of: muscle pH, purge loss, muscle color, firmness and marbling scores, intramuscular fat percentage, and tenderness.

41. The method of claim 1 wherein at least one selected trait is a reproductive trait selected from the group consisting of: number of piglets born per litter, piglet birth weight, piglet survival rate, pigs weaned per litter, litter weaning weight, age at puberty, farrowing rate, days to estrus, and semen quality.

42. The method of claim 1 wherein at least one selected trait is a health trait selected from the group consisting of: the absence of undesirable physical abnormalities or defects, improvement of feet and leg soundness, resistance to specific diseases or disease organisms, or general resistance to pathogens.

43. A target herd produced using a method according to either claim 1 or claim 33.

44. The herd of claim 43 wherein semen from the elite sire is used in a breeding plan suitable for producing an average of at least 160 half-sib offspring per week from the elite sire, for a plurality of successive weeks.

45. The herd of claim 43 wherein semen from the elite sire is used in a breeding plan is suitable for producing an average of at least 320 half-sib offspring per week from the elite sire, for a plurality of successive weeks.

46. The herd of claim 43 wherein semen from the elite sire is used in a breeding plan is suitable for producing an average of at least 640 half-sib offspring per week from the elite sire, for a plurality of successive weeks.

47. The herd of claim 43 wherein semen from the elite sire is used in a breeding plan is suitable for producing an average of at least 1280 half-sib offspring per week from the elite sire, for a plurality of successive weeks.

48. The target herd of claim 43 wherein the improved trait(s) comprise at least one trait selected from the group consisting of: efficient growth traits; meat quality traits; reproduction traits; and health traits.

49. The herd of claim 43 wherein at least one trait is an efficient growth trait selected from the group consisting of: average daily gain, average daily feed intake, feed efficiency, back fat thickness, loin muscle area, and lean percentage.

50. The herd of claim 43 wherein at least one trait is a meat quality trait selected from the group consisting of: muscle pH, purge loss, muscle color, firmness and marbling scores, intramuscular fat percentage, and tenderness.

51. The herd of claim 43 wherein at least one trait is a reproductive trait selected from the group consisting of: number of piglets born per litter, piglet birth weight, piglet survival rate, pigs weaned per litter, litter weaning weight, age at puberty, farrowing rate, days to estrus, and semen quality.

52. The herd of claim 43 wherein at least one trait is a health trait selected from the group consisting of: the absence of undesirable physical abnormalities or defects, improvement of feet and leg soundness, resistance to specific diseases or disease organisms, or general resistance to pathogens.

53. A swine production (SP) herd produced using terminal parental swine produced using the method of claim 1.

54. A genetic nucleus (GN) herd or a target herd produced using swine produced by the method of claim 1 as replacement animals.

55. The herd of claim 54 that is a genetic nucleus herd.

56. The herd of claim 54 that is a target herd.

57. The target herd of claim 56 selected from the group consisting of at least one of the following: a production nucleus herd, a crossbred terminal boar multiplier herd, a daughter nucleus herd, and a crossbred gilt multiplier herd.

58. The herd of claim 54 wherein semen from the elite sire is used in a breeding plan suitable for producing an average of at least 160 half-sib offspring per week from the elite sire, for a plurality of successive weeks.

59. The herd of claim 54 wherein semen from the elite sire is used in a breeding plan is suitable for producing an average of at least 320 half-sib offspring per week from the elite sire, for a plurality of successive weeks.

60. The herd of claim 54 wherein semen from the elite sire is used in a breeding plan is suitable for producing an average of at least 640 half-sib offspring per week from the elite sire, for a plurality of successive weeks.

61. The herd of claim 54 wherein semen from the elite sire is used in a breeding plan is suitable for producing an average of at least 1280 half-sib offspring per week from the elite sire, for a plurality of successive weeks

62. A Method for making rapid change in the frequency of an allele in a target herd while concomitantly maintaining optimal long-term selection pressure for the allele in a genetic nucleus (GN) herd, the method comprising:
a. providing at least one genetic nucleus herd at least one target herd;
b. selecting an allele for which increased frequency in the target herd is desired;
c. providing semen aliquots from an elite sire selected from a genetic nucleus (GN) herd wherein the elite sire has a desired germplasm that is determinative for increasing the frequency of the desired allele;
d. using the semen aliquots to impregnate a correlative number of breeding females in a target herd; wherein the semen from the elite sire is used to breed substantially all of the females in the target herd;
e. producing half-sib offspring having an increased frequency of the selected allele when compared with the breeding females in the target herd; and
f. repeating steps c. through e. as necessary to achieve the desired frequency for the selected allele;
wherein the inbreeding rate of the GN herd is maintained at an acceptable level.

63. The method of claim 62 wherein the selected elite sire is homozygous for the selected allele.

64. The method of claim 62 carried out so as to fix the selected allele in target herd.

65. The method of claim 62 where there is substantially no reduction in genetic variance in the GN herd, at the selected allele.

66. A method for producing swine offspring, the method comprising:
a. providing at least one genetic nucleus herd and/or a target herd for which improvement is desired
b. selecting a trait or traits, for which improvement is desired;
c. providing semen aliquots from an elite sire selected from the genetic nucleus (GN) herd wherein the elite sire has a desired germplasm that is determinative for improving one or more selected trait(s) in the target herd;
d. using the semen aliquots to impregnate a correlative number of breeding females in a target herd; wherein the semen from the elite sire is used to breed substantially all of the females in the target herd;
e. transferring embryos from the breeding females to surrogate carrier females; and
f. producing half-sib offspring having improved germplasm when compared with the breeding females in the target herd

67. The method of claim 66 wherein the embryos are transferred to surrogate carrier females in a specific pathogen free herd.

68. The method of claim 67 wherein the elite sir and/or the breeding female is not specific pathogen free.

69. The method of claim 66 wherein the protocol for choosing which elite sire and/or breeding females to mate employs marker-assisted selection and/or marker-assisted allocation.
